(19) United States
(12) Patent Application Publication
(10) Pub. No.: US 2004/0224313 A1
(43) Pub. Date: Nov. 11, 2004

(54) Polymorphisms in the human cyp2b6 gene and their use in diagnostic and therapeutic applications

(76) Inventors: Ulrich M Zanger, Korntal (DE); Thomas Lang, Weilheim der Teck (DE)

Correspondence Address: Jane T Gunnison, Fish & Neave, 125 Avenue of the Americas, New York, NY, 10020-1104, US

(21) Appl. No.: 09/958,635

(22) PCT Filed: Feb. 9, 2001
(86) PCT No.: PCT/EP01/01456

(30) Foreign Application Priority Data
Feb. 9, 2000 (EP) 00102701.0

(51) Int. Cl. C12Q 1/68

(52) U.S. Cl. 435/6

(57) ABSTRACT

Described are general means and methods of diagnosing and treating the phenotypic spectrum as well as the overlapping clinical characteristics with several forms of inherited abnormal expression and/or function of the CYP2B6 genes. In particular, polynucleotides of molecular variant CYP2B6 genes which, for example, are associated with insufficient metabolization and/or sensitivety of drugs, and vectors comprising such polynucleotides are provided. Furthermore, host cells comprising such polynucleotides or vectors and their use for the production of variant CYP2B6 proteins are described. In addition, variant CYP2B6 proteins and antibodies specifically recognizing such proteins as well as transgenic non-human animals comprising the above-described polynucleotide or vectors are provided. Described are also methods for identifying and obtaining inhibitors for therapy of disorders related to the malfunction of the CYP2B6 gene as well as methods of diagnosing the status of such disorders. Pharmaceutical and diagnostic compositions useful for diagnosing and treating various diseases with drugs that are substrates, inhibitors or modulators of the CYP2B6 gene product are described as well.

### FIELD OF THE INVENTION

The present invention relates generally to means and methods of diagnosing and treating the phenotypic spectrum as well as the overlapping clinical characteristics with several forms of inherited abnormal expression and/or function of the cytochrome P-450 (CYP)2B6 gene. In particular, the present invention relates to polynucleotides of molecular variant CYP2B6 genes which, for example, are associated with abnormal drug response or individual predisposition to several common diseases caused by environmental or individual exposure to substances such as carcinogens and nicotine, and to vectors comprising such polynucleotides. Furthermore, the present invention relates to host cells comprising such polynucleotides or vectors and their use for the production of variant CYP2B6 proteins. In addition, the present invention relates to variant CYP2B6 proteins and antibodies specifically recognizing such proteins. The present invention also concerns transgenic non-human animals comprising the above-described polynucleotide or vectors. Moreover, the present invention relates to methods for identifying and obtaining drug candidates and inhibitors for therapy of disorders related to the malfunction of the CYP2B6 genes as well as to methods of diagnosing the status of such disorders. The present invention furthermore provides pharmaceutical and diagnostic compositions comprising the above-described polynucleotides, vectors, proteins, antibodies, and drugs and inhibitors obtainable by the above-described method. Said compositions are particularly useful for diagnosing and treating various diseases with drugs that are substrates, inhibitors or modulators of CYP2B6 genes or their product.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND OF THE INVENTION

Members of the cytochrome P-450 (CYP) family of hemoproteins metabolise a wide variety of endogenous substrates such as steroid hormones, and of xenobiotics including carcinogens, toxins and drugs; see, e.g., Daly, Toxicol. Lett. 102-103 (1998), 143-147; Touw, Drug Metabol. Drug Interact. 14 (1997), 55-82. CYP2B6 is involved in the metabolic activation and inactivation of a number of clinically important drugs such as the frequently used antineoplastic agents cyclophosphamide and ifosfamide, the antioestrogen tamoxifen, the newer platelet aggregation inhibitor Clopidogrel, but also envirenmental toxicants and recreational drugs such as nicotine and ecstasy. The CYP2B6 gene has been mapped to chromosome 19 between 19q12 and 19q13.2 (Miles et al., 1989; Carrano et al., 1989; Hoffman et al., 1995). It spans a region of approximately 28 kb and like other members of the CYP2 family it contains 9 exons which are separated by 8 introns. The gene encodes a microsomal protein with 491 amino acids. The CYP2B6 gene is located within a 350 kb gene cluster together with a number of other genes and pseudogenes of the CYP2A, 2B, and 2F subfamilies (Hoffman et al., 1995). The CYP2B subfamily consists of the functional CYP2B6 gene, the nonfunctional CYP2B7 gene and a CYP2B7-like pseudogene (Yamano 1989; Hoffman 1995). CYP2B6 mRNA and protein are primarily expressed in human liver where it contributes an estimated 1 to 5% to the total liver content of cytochrome P450 (Gervot et al., 1999). Extrahepatic expression occurs at lower levels in kidney, intestine and lung (Gervot et al., 1999; Gonzalez et al., 1992). Initially, it was thought that CYP2B6 is expressed in only about 25% of all human livers (Mimura et al., 1993). However, with new antibodies of higher sensitivity CYP2B6 was detectable in all liver specimens with an interindividual variability of expression of up to 100-fold (Code et al., 1997; Gervot et al., 1999). The reasons for this high variability are unknown but most likely two mechanisms are involved:

(a) enzyme induction: CYP2B6 is the human homologe to the rat P450s CYP2B1/CYP2B2 which are known to be inducible by phenobarbital (Sueyoshi et al., 1999);

(b) genetic polymorphism: some limited indications for existing genetic polymorphisms, including a polymorphic Bam HI and Bgl II site observed as RFLP were described in one of the original publications (Miles et al., 1988; Yamano et al. 1989).

It is clear that naturally occurring mutations, if they exist can have effects on drug metabolization and efficacy of therapies with drugs, for example in cancer treatment. It is unknown, however, how many of such variations exist, and with what frequency and at what positions in the human CYP2B6 gene.

Accordingly, means and methods for diagnosing and treating a variety of forms of individual drug intolerability and inefficacy of drug therapy which result from CYP2B6 gene polymorphisms that interfere e.g., with chemotherapeutic treatment of diseases, was hitherto not available but are nevertheless highly desirable.

Thus, the technical problem of the present invention is to comply with the needs described above.

The solution to this technical problem is achieved by providing the embodiments characterized in the claims.

### SUMMARY OF THE INVENTION

The present invention is based on the finding of novel, so far unknown variations in the nucleotide sequence of the CYP2B6 gene and the population distribution of these alleles. Based upon the knowledge of these novel sequences diagnostic tests and reagents for such tests were designed for the specific detection and genotyping of CYP2B6 alleles in humans, including homozygous as well as heterozygous, frequent as well as rare alleles of the CYP2B6 genes. The determination of the CYP2B6 gene allele status of humans with such tests is useful for the optimization of therapies with the numerous substrates of CYP2B6. It may also be useful in the determination of the individual predisposition to several common cancers caused by environmental carcinogens.

In a first embodiment, the invention provides polynucleotides of molecular variant CYP2B6 genes, oligonucleotides useful for detecting such genes, and embodiments related thereto such as vectors, host cells, variant CYP2B6 proteins and methods for producing the same.

In yet another embodiment, the invention provides methods for identifying and obtaining drug candidates and inhibitors of CYP2B6 for therapy of disorders related to acquired drug hypo- or hypersensitivity as well as methods of diagnosing the status of such disorders.

In a further embodiment, the invention provides pharmaceutical and diagnostic compositions comprising the above-described polynucleotides, oligonucleotides, vectors, proteins, antibodies thereto, and drugs and inhibitors obtainable by the above-described method.

The pharmaceutical and diagnostic compositions, methods and uses of the invention are useful for the diagnosis and treatment of cancer and other diseases the therapy of which is dependent on drug treatment and tolerance. The novel variant forms of CYP2B6 genes according to the invention provide the potential for the development of a pharmacodynamic profile of drugs for a given patient.

### DESCRIPTION OF THE INVENTION

The finding and characterization of variations in the CYP2B6 gene, and diagnostic tests for the discrimination of different CYP2B6 alleles in human individuals provide a very potent tool for improving the therapy of diseases with drugs that are targets of the CYP2B6 gene product, and whose metabolization is therefore dependent on CYP2B6. The diagnosis of the individual allelic CYP2B6 status permits a more focused therapy, e.g., by opening the possibility to apply individual dose regimens of drugs. It may also be useful as prognostic tool for therapy outcome. Furthermore, diagnostic tests to genotype CYP2B6, and novel CYP2B6 variants, will not only improve therapy with established drugs and help to correlate genotypes with drug activity or side effects. These tests and sequences also provide reagents for the development of novel inhibitors that specifically modulate the activity of the individual types of CYP2B6. Expression in yeast, for example, of three allelic cDNAs encoding human liver CYP3A4 and methods for testing the binding properties and catalytic activities of their gene products have been described in Peyronneau, Eur. J. Biochem. 218 (1993).

Thus, the present invention provides a novel way to exploit molecular biology and pharmacological research for drug therapy while bypassing their potential detrimental effects which are due to expression of variant CYP2B6 genes.

Accordingly, the invention relates to a polynucleotide comprising a polynucleotide selected from the group consisting of:

(a) a polynucleotide having the nucleic acid sequence of any one of SEQ ID NO: 44 to 59;

(b) a polynucleotide encoding a polypeptide having the amino acid sequence of any one of SEQ ID NO: 60 to 64;

(c) a polynucleotide capable of hybridizing to a molecular variant of the cytochrome P450 (CYP)2B6 gene, wherein said polynucleotide is having at a position corresponding to position −1777, −1455, −1185, −750 or −82 of the CYP2B6 gene (accession No: AC023172, wherein nucleotide C at position 4115 has been numbered −1), at a position corresponding to position −18 of the CYP2B6 gene (accession No: AC023172, wherein nucleotide G at position 19714 has been numbered −1), at a position corresponding to position 59 of the CYP2B6 gene (accession No: AC023172, wherein nucleotide G at position 16974 has been numbered +1), or at a position corresponding to position 64, 78, 216, 516, 714, 732, 777, 785, or 1459 of the CYP2B6 gene (accession No: M29874, wherein nucleotide A at position 7 has been numbered +1), at least one nucleotide substitution, deletion and/or addition;

(d) a polynucleotide capable of hybridizing to a molecular variant of the cytochrome P450 (CYP)2B6 protein, wherein said polynucleotide is having at a position corresponding to position −18 of the CYP2B6 gene (accession No: AC023172, wherein nucleotide G at position 19714 has been numbered −1), at a position corresponding to position 59 of the CYP2B6 gene (accession No: AC023172, wherein nucleotide G at position 16974 has been numbered +1) or at a position corresponding to position 64, 78, 516, 732, or 1459 of the CYP2B6 gene (accession No: M29874, wherein nucleotide A at position 7 has been numbered +1) a T, at a position corresponding to position 785 of the CYP2B6 gene (accession No: M29874, wherein nucleotide A at position 7 has been numbered +1) or at a position corresponding to position −1185 or −1777 of the CYP2B6 gene (accession No: AC023172, wherein nucleotide C at position 4115 has been numbered −1) a G, at a position corresponding to position −1455, −750, −82 of the CYP2B6 gene (accession No: AC023172, wherein nucleotide C at position 4115 has been numbered −1) or at a position corresponding to position 216 of the CYP2B6 gene (accession No: M29874, wherein nucleotide A at position 7 has been numbered +1) a C or at a position corresponding to position 714 or 777 of the CYP2B6 gene (accession No: M29874, wherein nucleotide A at position 7 has been numbered +1) an A;

(e) a polynucleotide encoding a CYP2B6 polypeptide or fragment thereof, wherein said polypeptide comprises at least one amino acid deletion, addition and/or substitution at an amino acid position corresponding to amino acid residue Arg22 in exon 1, Gln172 in exon 4, Ser259 and/or Lys262 in exon 5 and/or Arg487 in exon 9 of the CYP2B6 polypeptide (accession No: M29874);

(f) a polynucleotide encoding a CYP2B6 polypeptide or fragment thereof, wherein said polypeptide comprises an amino acid substitution of Arg22 to Cys in exon 1, Gln172 to His in exon 4, Ser259 to Arg and Lys262 to Arg in exon 5 and/or Arg487 to Cys in exon 9 of the CYP2B6 polypeptide (accession No: M29874);

(g) a polynucleotide comprising a nucleotide sequence which is not cleaved by the restriction endonuclease HaeII and which is obtainable by PCR amplification from human genomic DNA using oligonucleotides having the SEQ ID NO: 1 and 2 as primers, wherein said polynucleotide is capable of hybridizing to the CYP2B6 gene;

(h) a polynucleotide comprising a nucleotide sequence which is cleaved by the restriction endonuclease BsrI one time and which is obtainable by PCR amplification from human genomic DNA using oligonucleotides having the SEQ ID NO: 5 and 6 as primers, wherein said polynucleotide is capable of hybridizing to the CYP2B6 gene;

(i) a polynucleotide comprising a nucleotide sequence which is cleaved by the restriction endonuclease HaeII one time and which is obtainable by PCR amplification from human genomic DNA using oligonucleotides having the SEQ ID NO: 9 and 10 as primers, wherein said polynucleotide is capable of hybridizing to the CYP2B6 gene;

(j) a polynucleotide comprising a nucleotide sequence which is cleaved by the restriction endonuclease StyI two times and which is obtainable by PCR amplification from human genomic DNA using oligonucleotides having the SEQ ID NO: 9 and 10 as primers, wherein said polynucleotide is capable of hybridizing to the CYP2B6 gene;

(k) a polynucleotide comprising a nucleotide sequence which is cleaved by the restriction endonuclease Bgl II one time and which is obtainable by PCR amplification from human genomic DNA using oligonucleotides having the SEQ ID NO: 13 and 14 as primers, wherein said polynucleotide is capable of hybridizing to the CYP2B6 gene;

(l) a polynucleotide comprising a nucleotide sequence which is cleaved by the restriction endonuclease BspHI one time and which is obtainable by PCR amplification from human genomic DNA using oligonucleotides having the SEQ ID NO: 1 and 2 as primers, wherein said polynucleotide is capable of hybridizing to the CYP2B6 gene;

(m) a polynucleotide comprising a nucleotide sequence which is cleaved by the restriction endonuclease EcoRII nine times and which is obtainable by PCR amplification from human genomic DNA using oligonucleotides having the SEQ ID NO: 2 and 3 as primers, wherein said polynucleotide is capable of hybridizing to the CYP2B6 gene;

(n) a polynucleotide comprising a nucleotide sequence which is not cleaved by the restriction endonuclease PstI and which is obtainable by PCR amplification from human genomic DNA using oligonucleotides having the SEQ ID NO: 9 and 10 as primers, wherein said polynucleotide is capable of hybridizing to the CYP2B6 gene;

(o) a polynucleotide comprising a nucleotide sequence which is cleaved by the restriction endonuclease BceFI two times and which is obtainable by PCR amplification from human genomic DNA using oligonucleotides having the SEQ ID NO: 42 and 43 as primers, wherein said polynucleotide is capable of hybridizing to the CYP2B6 gene;

(p) a polynucleotide comprising a nucleotide sequence which is cleaved by the restriction endonuclease AccI one time and which is obtainable by PCR amplification from human genomic DNA using oligonucleotides having the SEQ ID NO: 42 and 43 as primers, wherein said polynucleotide is capable of hybridizing to the CYP2B6 gene;

(q) a polynucleotide comprising a nucleotide sequence which is cleaved by the restriction endonuclease AvaII six times and which is obtainable by PCR amplification from human genomic DNA using oligonucleotides having the SEQ ID NO: 42 and 43 as primers, wherein said polynucleotide is capable of hybridizing to the CYP2B6 gene; and

(r) a polynucleotide comprising a nucleotide sequence which is cleaved by the restriction endonuclease MseI nine times and which is obtainable by PCR amplification from human genomic DNA using oligonucleotides having the SEQ ID NO: 42 and 43 as primers, wherein said polynucleotide is capable of hybridizing to the CYP2B6 gene;

The variant polynucleotides and polypeptides also comprise fragments of said polynucleotides or polypeptides of the invention. The polynucleotides and polypeptides as well as the aforementioned fragments thereof are characterized as being associated with altered biochemical properties of the CYP2B6 protein and/or altered expression of the variant CYP2B6 gene compared to the corresponding wild type gene. Said altered biochemical properties are considered within the gist of the present invention to lead to altered biochemical properties of the CYP2B6 protein such as protein stability, activity, or substrate specificity and therefore are considered to lead to interindividual differences in drug metabolism.

In the context of the present invention the term “molecular variant” CYP2B6 gene or protein as used herein means that said CYP2B6 gene or protein differs from the wild type CYP2B6 gene or protein by way of nucleotide substitution(s), addition(s) and/or deletion(s) (partly genomic and cDNA sequences of the CYP2B6 gene are described in, for example Miles et al., 1988; Miles et al.,1989; Miles et al., 1990; Yamano et al., 1989; Sueyoshi et al.,1999; accession numbers:J02864; X13494; M29874; X06399; X16864; X06400; Af081569). Preferably, said nucleotide substitution(s) result(s) in a corresponding change in the amino acid sequence of the CYP2B6 protein.

The term “hybridizing” as used herein refers to polynucleotides which are capable of hybridizing to the polynucleotides of the invention or parts thereof which are associated with altered biochemical properties of the CYP2B6 protein and/or altered expression of the variant CYP2B6 gene compared to the corresponding wild type gene. Thus, said hybridizing polynucleotides are also associated with said altered biochemical properties of the CYP2B6 protein and/or said altered expression of the variant CYP2B6 gene compared to the corresponding wild type gene. Therefore, said polynucleotides may be useful as probes in Northern or Southern Blot analysis of RNA or DNA preparations, respectively, or can be used as oligonucleotide primers in PCR analysis dependent on their respective size. Also comprised by the invention are hybridizing polynucleotides which are useful for analysing DNA-Protein interactions via, e.g., electrophoretic mobility shift analysis (EMSA). Preferably, said hybridizing polynucleotides comprise at least 10, more preferably at least 15 nucleotides in length while a hybridizing polynucleotide of the present invention to be used as a probe preferably comprises at least 100, more preferably at least 200, or most preferably at least 500 nucleotides in length. It is well known in the art how to perform hybridization experiments with nucleic acid molecules, i.e. the person skilled in the art knows what hybridization conditions s/he has to use in accordance with the present invention. Such hybridization conditions are referred to in standard text books such as Molecular Cloning A Laboratory Manual, Cold Spring Harbor Laboratory (1989) N.Y. Preferred in accordance with the present inventions are polynucleotides which are capable of hybridizing to the polynucleotides of the invention or parts thereof which are associated with altered biochemical properties of the CYP2B6 protein and/or altered expression of the variant CYP2B6 gene compared to the corresponding wild type gene under stringent hybridization conditions, i.e. which do not cross hybridize to unrelated polynucleotides such as polynucleotides that may not alter biochemical properties of the CYP2B6 protein and/or alter expression of the variant CYP2B6 gene compared to the corresponding wild type gene.

The term “corresponding” as used herein means that a position is not only determined by the number of the preceding nucleotides and amino acids, respectively. The position of a given nucleotide or amino acid in accordance with the present invention which may be deleted, substituted or comprise one or more additional nucleotide(s) may vary due to deletions or additional nucleotides or amino acids elsewhere in the promotor and/or gene. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as functional domains or motifs of the protein of the invention.

In accordance with the present invention, the mode and population distribution of novel so far unidentified genetic variations in the CYP2B6 gene have been analyzed by sequence analysis of relevant regions of the human CYP2B6 gene from many different individuals. Except for some limited initial observations which indicated the occurrence of genetic polymorphisms of the CYP2B6 gene (Miles et al., 1989; Yamano et al., 1989), no systematic analysis has been carried out. This study was undertaken in accordance with the present invention as the first systematic approach to detect genetic polymorphisms in the human CYP2B6 gene and, surprisingly, revealed that mutations in the CYP2B6 gene exist, in particular in the coding regions of the gene that can be expected to cosegregnate and optionally lead to altered biochemical properties of the CYP2B6 protein such as protein stability, activity, or substrate specificity and will lead to interindividual differences in drug metabolism. It is a well known fact that genomic DNA of individuals, which harbor the individual genetic makeup of all genes, including CYP2B6 can easily be purified from individual blood samples. These individual DNA samples are then used for the analysis of the sequence composition of the CYP2B6 gene alleles that are present in the individual which provided the blood sample. The sequence analysis was carried out by PCR amplification of relevant regions of the CYP2B6 gene, subsequent purification of the PCR products, followed by automated DNA sequencing with established methods; see the Examples. One important parameter that had to be considered in the attempt to determine the individual CYP2B6 genotype and identify novel CYP2B6 variants by direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations. The details of the different steps in the identification and characterization of novel CYP2B6 gene polymorphisms (homozygous and heterozygous) are described in the examples below.

The mutations in the CYP2B6 genes detected in accordance with the present invention are illustrated in FIG. 1 and 2 and in Tables 2 and 3, respectively. The methods of the mutation analysis followed standard protocols and are described in detail in the examples. In general such methods to be used in accordance with the present invention for evaluating the phenotypic spectrum as well as the overlapping clinical characteristics with other forms of drug metabolization and altered tolerance to drugs in patients with mutations in the CYP2B6 gene encompass for example haplotype analysis, single-strand conformation polymorphism analysis (SSCA), PCR and direct sequencing. On the basis of thorough clinical characterization of many patients the phenotypes can then be correlated to these mutations as well as to mutations that had been described earlier for other CYPs.

As is evident to the person skilled in the art this new molecular genetic knowledge can now be used to exactly characterize the genotype of the index patient where a given drug takes an unusual effect and of his family.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient, for example, and characterization of disease (Bertz, Clin. Pharmacokinet. 32 (1997), 210-256; Engel, J. Chromatogra. B. Biomed. Appl. 678 (1996), 93-103). For the providers of health care, such as health maintenance organizations in the US and government public health services in many European countries, this pharmacogenomics approach can represent a way of both improving health care and reducing overheads because there is a large cost to unnecessary therapies, ineffective drugs and drugs with side effects.

The mutations in the variant CYP2B6 gene result in amino acid deletion(s), insertion(s) and In particular in substitution(s) either alone or in combination. It is of course also possible to genetically engineer such mutations in wild type genes or other mutant forms. Methods for introducing such modifications in the DNA sequence of CYP2B6 gene are well known to the person skilled in the art; see, e.g., Sambrook, Molecular Cloning A Laboratory Manual, Cold Spring Harbor Laboratory (1989) N.Y.

In a preferred embodiment of the invention, the above described polynucleotide encodes a variant CYP2B6 protein or fragment thereof, e.g., comprising one or more epitopes of the amino acid sequence encoded by the polynucleotide of (b) defined above.

For the investigation of the nature of the alterations in the amino acid sequence of the CYP2B6 proteins computer programs may be used such as BRASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analysis can be used for the identification of the influence of a particular mutation on binding and/or metabolization of drugs.

Usually, said amino acid deletion, addition or substitution in the amino acid sequence of the protein encoded by the polynucleotide of the invention is due to one or more nucleotide substitution, insertion or deletion, or any combinations thereof. Preferably said nucleotide substitution, insertion or deletion results in an amino acid substitution of Arg22 to Cys in exon 1, Gln172 to His in exon 4, Ser259 to Arg and Lys262 to Arg in exon 5 and/or Arg487 to Cys in exon 9 of the CYP2B6 gene.

The polynucleotide of the invention may further comprise at least one nucleotide and optionally amino acid deletion, addition and/or substitution in its encoded protein other than those specified hereinabove. This embodiment of the present invention allows the study of synergistic effects of the mutations in the CYP gene on the pharmacological profile of drugs in patients who bear such mutant forms of these genes or similar mutant forms that can be mimicked by the above described proteins. It is expected that the analysis of said synergistic effects provides deeper insights into drug tolerant or sensitive phenotypes of certain forms of cancer and other diseases. From said deeper insight the development of diagnostic and pharmaceutical compositions related to cancer will greatly benefit.

In another preferred embodiment, the present invention relates to polynucleotides of molecular variant CYP2B6 gene, wherein the nucleotide deletion, addition and/or substitution result in altered expression of the variant CYP2B6 gene compared to the corresponding wild type gene. A corresponding mutation can be found in the promoter region of the gene or in an intron.

In another embodiment the invention relates to a polynucleotide comprising a nucleotide sequence of at least one promoter, intron and/or exon of a variant cytochrome P450 (CYP)2B6 gene, wherein said promoter, intron or exon differs from that of the corresponding wild type gene by way of at least one nucleotide substitution, deletion and/or addition. Another embodiment of the present invention relates to polynucleotides comprising a nucleotide sequence encoding a molecular variant of the cytochrome P450 (CYP)2B6 protein by way of at least one amino acid deletion, addition and/or substitution at an amino acid position corresponding amino acid residue Arg22 in exon 1, Gln172 in exon 4, Ser259 and/or Lys262 in exon 5 and/or Arg487 in exon 9 of the CYP2B6 gene. In another embodiment the present invention relates to polynucleotides comprising a nucleotide sequence encoding a CYP2B6 polypeptide or fragment thereof having an epitope comprising an amino acid sequence encoded by the nucleotide sequence as defined above.

The polynucleotide of the invention may be, e.g., DNA, cDNA, genomic DNA, RNA or synthetically produced DNA or RNA or a recombinantly produced chimeric nucleic acid molecule comprising any of those polynucleotides either alone or in combination. Preferably said polynucleotide is part of a vector, particularly plasmids, cosmids, viruses and bacteriophages used conventionally in genetic engineering that comprise a polynucleotide of the invention. Such vectors may comprise further genes such as marker genes which allow for the selection of said vector in a suitable host cell and under suitable conditions.

In a further preferred embodiment of the vector of the invention, the polynucleotide of the invention is operatively linked to expression control sequences allowing expression in prokaryotic or eukaryotic cells. Expression of said polynucleotide comprises transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells.

The present invention furthermore relates to host cells transformed with a polynucleotide or vector of the invention. Said host cell may be a prokaryotic or eukaryotic cell; see supra. The polynucleotide or vector of the invention which is present in the host cell may either be integrated into the genome of the host cell or it may be maintained extrachromosomally. In this respect, it is also to be understood that the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a polynucleotide for the expression of a variant CYP2B6 protein or fragment thereof. Prokaryotic hosts may include gram negative as well as gram positive bacteria such as, for example, E. coli, S. typhimurium, Serratia marcescens and Bacillus subtilis. A polynucleotide coding for a mutant form of CYP2B6 variant proteins can be used to transform or transfect the host using any of the techniques commonly known to those of ordinary skill in the art. Methods for preparing fused, operably linked genes and expressing them in bacteria or animal cells are well-known in the art (Sambrook, supra). The genetic constructs and methods described therein can be utilized for expression of variant CYP2B6 proteins in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The proteins of the invention can then be isolated from the grown medium, cellular lysates, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies.

Thus, in a further embodiment the invention relates to a method for the production of variant CYP2B6 proteins and fragments thereof comprising culturing a host cell as defined above under conditions allowing the expression of the protein and recovering the produced protein or fragment from the culture.

In another embodiment the present invention relates to a method for producing cells capable of expressing a variant CYP2B6 gene comprising genetically engineering cells with the polynucleotide or with the vector of the invention. The cells obtainable by the method of the invention can be used, for example, to test drugs according to the methods described in Sambrook, Fritsch, Maniatis (1989). Molecular cloning: a laboratory manual. Cold Spring Harbour Laboratory press, Cold Spring Harbour; Peyronneau, Eur J Biochem 218 (1993), 355-61; Yamazaki, Carcinogenesis 16 (1995), 2167-2170. Furthermore, the cells can be used to study known drugs and unknown derivatives thereof for their ability to complement loss of drug efficacy caused by mutations in the CYP2B6 gene. For these embodiments the host cells preferably lack a wild type allele, preferably both alleles of the CYP2B6 gene and/or have at least one mutated from thereof. Alternatively, strong overexpression of a mutated allele over the normal allele and comparison with a recombinant cell line overexpressing the normal allele at a similar level may be used as a screening and analysis system. The cells obtainable by the above-described method may also be used for the screening methods referred to herein below.

Furthermore, the invention relates to variant CYP2B6 proteins and fragments thereof encoded by a polynucleotide according to the invention or obtainable by the above-described methods or from cells produced by the method described above. In this context it is also understood that the variant CYP2B6 proteins according to the invention may be further modified by conventional methods known in the art. By providing the variant CYP2B6 proteins according to the present invention it is also possible to determine the portions relevant for their biological activity or inhibition of the same.

The present invention furthermore relates to antibodies specifically recognizing a variant CYP2B6 protein according to the invention. Advantageously, the antibody specifically recognizes an epitope containing one or more amino acid substitution(s) as defined above.

Antibodies against the variant CYP2B6 protein of the invention can be prepared by well known methods using a purified protein according to the invention or a (synthetic) fragment derived therefrom as an antigen. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals. The antibodies can be monoclonal antibodies, polyclonal antibodies or synthetic antibodies as well as fragments of antibodies, such as Fab, Fv or scFv fragments etc. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant CYP2B6 proteins of the invention as well as for the monitoring of the presence of such variant CYP2B6 protein, for example, in transgenic organisms, and for the identification of compounds interacting with the proteins according to the invention. For example, surface plasmon resonance as employed in the BIAcore system can be used to increase the efficiency of phage antibodies which bind to an epitope of the protein of the invention (Schier, Human Antibodies Hybridomas 7 (1996), 97-105; Malmborg, J. Immunol. Methods 183 (1995), 7-13).

Furthermore, the present invention relates to nucleic acid molecules which represent or comprise the complementary strand of any of the above described polynucleotides or a part thereof, thus comprising at least one nucleotide difference compared to the corresponding wild type CYP2B6 gene nucleotide sequences specified by the above described nucleotide substitutions, deletions and additions. Such a molecule may either be a deoxyribonucleic acid or a ribonucleic acid. Such molecules comprise, for example, antisense RNA. These molecules may furthermore be linked to sequences which when transcribed code for a ribozyme thereby producing a ribozyme which specifically cleaves transcripts of polynucleotides according to the invention.

Furthermore, the present invention relates to a vector comprising a nucleic acid molecule according to the invention. Examples for such vectors are described above. Preferably, the nucleic acid molecule present in the vector is operatively linked to regulatory elements permitting expression in prokaryotic or eukaryotic host cells; see supra.

The present invention also relates to a method for the production of a transgenic non-human animal, preferably transgenic mouse, comprising introduction of a polynucleotide or vector of the invention into a germ cell, an embryonic cell, stem cell or an egg or a cell derived therefrom. The non-human animal can be used in accordance with the method of the invention described below and may be a non-transgenic healthy animal, or may have a disorder, preferably a disorder caused by at least one mutation in the CYP2B6 gene. Such transgenic animals are well suited for, e.g., pharmacological studies of drugs in connection with variant forms of the above described variant CYP2B6 proteins since these proteins or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe.

The invention also relates to transgenic non-human animals such as transgenic mouse, rats, hamsters, dogs, monkeys, rabbits, pigs, C. elegans and fish such as torpedo fish comprising a polynucleotide or vector of the invention or obtained by the method described above, preferably wherein said polynucleotide or vector is stably integrated into the genome of said non-human animal, preferably such that the presence of said polynucleotide or vector leads to the expression of the variant CYP2B6 gene of the invention. It may have one or several copies of the same or different polynucleotides of the variant CYP2B6 gene. This animal has numerous utilities, including as a research model for drug tolerability and therefore, presents a novel and valuable animal in the development of therapies, treatment, etc. for diseases caused by deficiency or failure of drug metabolization in the cell. Accordingly, in this instance, the mammal is preferably a laboratory animal such as a mouse or rat.

Preferably, the transgenic non-human animal of the invention further comprises at least one inactivated wild type allele of the CYP2B6 gene. This embodiment allows for example the study of the interaction of various variant forms of CYP2B6 proteins. It might be also desirable to inactivate CYP2B6 gene expression or function at a certain stage of development and/or life of the transgenic animal. This can be achieved by using, for example, tissue specific, developmental and/or cell regulated and/or inducible promoters which drive the expression of, e.g., an antisense or ribozyme directed against the RNA transcript of the CYP2B6 gene; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of the variant CYP2B6 gene may be controlled by such regulatory elements.

With the variant CYP2B6 polynucleotides and proteins and vectors of the invention, it is now possible to study in vivo and in vitro the efficiency of drugs in relation to particular mutations in the CYP2B6 gene of a patient and the affected phenotype. Furthermore, the variant CYP2B6 proteins of the invention can be used to determine the pharmacological profile of drugs and for the identification and preparation of further drugs which may be more effective for the treatment of, e.g., cancer, in particular for the amelioration of certain phenotypes caused by the respective mutations such as those described above.

Thus, a particular object of the present invention concerns drug/pro-drug selection and formulation of pharmaceutical compositions for the treatment of diseases which are amenable to chemotherapy taking into account the polymorphism of the variant form of the CYP2B6 gene that cosegregates with the affected phenotype of the patient to be treated. This allows the safe and economic application of drugs which for example were hitherto considered not appropriate for therapy of, e.g., cancer due to either their side effects in some patients and/or their unreliable pharmacological profile with respect to the same or different phenotype(s) of the disease. The means and methods described herein can be used, for example, to improve dosing recommendations and allows the prescriber to anticipate necessary dose adjustments depending on the considered patient group.

In a further embodiment the present invention relates to a method of identifying and obtaining a CYP2B6 inhibitor capable of modulating the activity of a molecular variant of the CYP2B6 gene or its gene product comprising the steps of

(a) contacting the variant CYP2B6 protein or a cell expressing a molecular variant gene comprising a polynucleotide of the invention in the presence of components capable of providing a detectable signal in response to drug metabolization, with a compound to be screened under conditions to permit CYP2B6 mediated drug metabolization, and

(b) detecting the presence or absence of a signal or increase of a signal generated from the metabolized drug, wherein the presence or increase of the signal is indicative for a putative inhibitor.

The term “compound” in a method of the invention includes a single substance or a plurality of substances which may or may not be identical.

Said compound(s) may be chemically synthesized or produced via microbial fermentation but can also be comprised in, for example, samples, e.g., cell extracts from, e.g., plants, animals or microorganisms. Furthermore, said compounds may be known in the art but hitherto not known to be useful as an inhibitor, respectively. The plurality of compounds may be, e.g., added to the culture medium or injected into a cell or non-human animal of the invention.

If a sample containing (a) compound(s) is identified in the method of the invention, then it is either possible to isolate the compound from the original sample identified as containing the compound, in question or one can further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described herein or in the literature (e.g. Yanev, Drug Metab. Dispos. 27 (1999), 600-604; Kobayashi, Drug Metab. Dispos. 27 (1999), 1429-1433; Kumar, Drug. Metab. Dispos. 27 (1999), 902-908; Ekins, Pharmacogenetics 7 (1997), 165-179; Heyn, Drug Metab. Dispos. 24 (1996), 948-954). Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art or by using and modifying the methods as described herein. Furthermore, the person skilled in the art will readily recognize which further compounds and/or enzymes may be used in order to perform the methods of the invention, for example, enzymes, if necessary, that convert a certain compound into the precursor which in turn represents a substrate for the CYP2B6 protein. Such adaptation of the method of the invention is well within the skill of the person skilled in the art and can be performed without undue experimentation.

Suitable assays which can be employed in accordance with the present invention are described, for example, in Hashimoto, Eur J Biochem 218 (1993), 585-95 wherein transfection assays with chimeric CYP3A4 genes in HepG2 cells are described. Similarly, the variant CYP2B6 gene can be expressed or co-expressed in HepG2 cells and analyzed for its transcriptional activity and catalytic properties of CYP2B6. Such an assay can also be used for studying the catalytic properties of the CYP2B6 on its substrates such as Cyclophosphamide, (Chang et al,1993); Iphosphamide (Granvil et al., 1999), Tamoxifen (Gillam et al., 1999), S-Mephenytoin (Heyn et al., 1996),. ) MDMA (N-methyl-3,4-methylenedioxyamphetamine, “Ecstasy”) and MDE (N-ethyl-3,4-methylenedioxyamphetamine, “Eve”). In particular, such tests are useful to add in predicting whether a given drug will interact in an individual carrying the respective variant CYP2B6 gene. In addition heterologous expression systems such as yeast can be used in order to study the stability, binding properties and catalytic activities of the gene products of the variant CYP2B6 genes compared to the corresponding wild type gene product. As mentioned before, the molecular variant CYP2B6 genes and their gene products, particularly when employed in the above described methods, can be used for pharmacological and toxicological studies of the metabolism of drugs. Preferred drugs to be tested in accordance with the methods of the present invention comprise those described in Table 4 below, but are not limited thereto. Studies with cDNA-expressed CYP2B6 have identified further drugs for this P450, including S-mephobarbital (Kobayashi et al.,1999), 7-ethoxy-4-trifluoromethylcoumarin (Code et al., 1997), 2,4,5,2′,4′,5′-hexachlorobiphenyl (Ariyoshi et al., 1995).

Compounds which can be used in accordance with the present invention include peptides, proteins, nucleic acids, antibodies, small organic compounds, ligands, peptidomimetics, PNAs and the like. Said compounds can also be functional derivatives or analogues of known drugs such as from those described above. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described. Furthermore, peptide mimetics and/or computer aided design of appropriate drug derivatives and analogues can be used, for example, according to the methods described below. Such analogs comprise molecules having as the basis structure of known CYP2B6-substrates and/or inhibitors and/or modulators; see infra.

Appropriate computer programs can be used for the identification of interactive sites of a putative inhibitor and the CYP2B6 protein of the invention by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors and the CYP2B6 protein of the invention can be used for the design of peptidomimetic drugs (Rose, Biochemistry 35 (1996), 12933-12944; Rutenberg, Bioorg. Med. Chem. 4 (1996),1545-1558).

In summary, the present invention provides methods for identifying and obtaining compounds which can be used in specific doses for the treatment of specific forms of diseases, e.g., cancer the chemotherapy of which is complicated by malfunctions of the CYP2B6 gene often resulting in an altered activity or level of drug metabolization or sensitive phenotype.

In a preferred embodiment of the method of the invention said cell is a cell of or, obtained by the method of the invention or is comprised in the above-described transgenic non-human animal.

In a further embodiment the present invention relates to a method of identifying and obtaining an CYP2B6 inhibitor capable of modulating the activity of a molecular variant of the CYP2B6 gene or its gene product comprising the steps of

(a) contacting the variant CYP2B6 protein of the invention with a first molecule known to be bound by wild type CYP2B6 protein to form a first complex of said protein and said first molecule;

(b) contacting said first complex with a compound to be screened; and

(c) measuring whether said compound displaces said first molecule from said first complex.

Advantageously, in said method said measuring step comprises measuring the formation of a second complex of said protein and said inhibitor candidate. Preferably, said measuring step comprises measuring the amount of said first molecule that is not bound to said protein.

In a particularly preferred embodiment of the above-described method said first molecule is for example Cyclophosphamide, Iphosphamide, Tarnoxifen, Clopidogrel and Orphenadrine. Furthermore, it is preferred that in the method of the invention said first molecule is labeled, e.g., with a radioactive or fluorescent label.

In a still further embodiment the present invention relates to a method of diagnosing a disorder related to the presence of a molecular variant CYP2B6 gene or susceptibility to such a disorder comprising

(a) determining the presence of a polynucleotide of the invention in a sample from a subject; and/or

(b) determining the presence of a variant form of CYP2B6 protein, for example with the antibody of the invention.

In accordance with this embodiment of the present invention, the method of testing the status of a disorder or susceptibility to such a disorder can be effected by using a polynucleotide or a nucleic acid molecule of the invention, e.g., in the form of a Southern or Northern blot or in situ analysis. Said nucleic acid sequence may hybridize to a coding region of either of the genes or to a non-coding region, e.g. intron. In the case that a complementary sequence is employed in the method of the invention, said nucleic acid molecule can again be used in Northern blots. Additionally, said testing can be done in conjunction with an actual blocking, e.g., of the transcription of the gene and thus is expected to have therapeutic relevance. Furthermore, a primer or oligonucleotide can also be used for hybridizing to one of the above-mentioned-CYP2B6 genes or corresponding mRNAs. The nucleic acids used for hybridization can, of course, be conveniently labeled by incorporating or attaching, e.g., a radioactive or other marker. Such markers are well known in the art. The labeling of said nucleic acid molecules can be effected by conventional methods.

Additionally, the presence or expression of variant CYP2B6 genes can be monitored by using a primer pair that specifically hybridizes to either of the corresponding nucleic acid sequences and by carrying out a PCR reaction according to standard procedures. Specific hybridization of the above mentioned probes or primers preferably occurs at stringent hybridization conditions. The term “stringent hybridization conditions” is well known in the art; see, for example, Sambrook et al., “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985. Furthermore, the mRNA, cRNA, cDNA or genomic DNA obtained from the subject may be sequenced to identify mutations which may be characteristic fingerprints of mutations in the CYP2B6 gene. The present invention further comprises methods wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments as described above.

Further modifications of the above-mentioned embodiment of the invention can be easily devised by the person skilled in the art, without any undue experimentation from this disclosure; see, e.g., the examples. An additional embodiment of the present invention relates to a method wherein said determination is effected by employing an antibody of the invention or fragment thereof. The antibody used in the method of the invention may be labeled with detectable tags such as a histidine flags or a biotin molecule.

In a preferred embodiment of the present invention, the above described methods comprise PCR, ligase chain reaction, restriction digestion, direct sequencing, nucleic acid amplification techniques, hybridization techniques or immunoassays (Sambrook et al., loc. cit. CSH cloning, Harlow and Lane loc. cit. CSH antibodies).

In a preferred embodiment of the method of the present invention said disorder is cancer.

In a further embodiment of the above-described method, a further step comprising administering to the subject a medicament to abolish or alleviate said variations in the CYP2B6 gene in accordance with all applications of the method of the invention allows treatment of a given disease before the onset of clinical symptoms due to the phenotype response caused by the CYP2B6 gene.

In a preferred embodiment of the method of the invention said medicament are chemotherapeutic agents such as substrates of CYP2B6, e.g., Cyclophosphamide, Iphosphamide,Tamoxifen,Clopidogrel.

In another preferred embodiment of the above-described methods, said method further comprises introducing

(i) a functional and expressible wild type CYP2B6 gene or

(ii) a nucleotide acid molecule or vector of the invention into cells.

In this context and as used throughout this specification, “functional” CYP2B6 gene means a gene wherein the encoded protein having part or all of the primary structural conformation of the wild type CYP2B6 protein, i.e. possessing the biological property of metabolizing drugs. This embodiment of the present invention is suited for therapy of, e.g., cancer in particular in humans. Detection of the expression of a variant CYP2B6 gene would allow the conclusion that said expression is interrelated to the generation or maintenance of a corresponding phenotype of the disease. Accordingly, a step would be applied to reduce the expression level to low levels or abolish the same. This can be done, for example, by at least partial elimination of the expression of the mutant gene by biological means, for example, by the use of ribozymes, antisense nucleic acid molecules, intracellular antibodies or the above described inhibitors against the variant forms of these CYP2B6 proteins. Furthermore, pharmaceutical products may be developed that reduce the expression levels of the corresponding mutant proteins and genes.

In a further embodiment the invention relates to a method for the production of a pharmaceutical composition comprising the steps of any one of the above described methods and synthesizing and/or formulating the compound identified in step (b) or a derivative or homologue thereof in a pharmaceutically acceptable form. The therapeutically useful compounds identified according to the method of the invention may be formulated and administered to a patient as discussed above. For uses and therapeutic doses determined to be appropriate by one skilled in the art see infra.

Furthermore, the present invention relates to a method for the preparation of a pharmaceutical composition comprising the steps of the above-described methods; and formulating a drug or pro-drug in the form suitable for therapeutic application and preventing or ameliorating the disorder of the subject diagnosed in the method of the invention. Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or inhibitor identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329).

In a preferred embodiment of the method of the present invention said drug or prodrug is a derivative of a medicament as defined hereinbefore.

In a still further embodiment the present invention relates to an inhibitor identified or obtained by the method described hereinbefore. Preferably, the inhibitor binds specifically to the variant CYP2B6 protein of the invention. The antibodies, nucleic acid molecules and inhibitors of the present invention preferably have a specificity at least substantially identical to binding specificity of the natural ligand or binding partner of the CYP2B6 protein of the invention. An antibody or inhibitor can have a binding affinity to the CYP2B6 protein of the invention of at least 10⁵ M⁻¹, preferably higher than 10⁷ M⁻¹ and advantageously up to 10¹⁰ M⁻¹ in case CYP2B6 activity should be repressed. Hence, in a preferred embodiment, a suppressive antibody or inhibitor of the invention has an affinity of at least about 10⁻⁷ M, preferably at least about 10⁻⁹ M and most preferably at last about 10⁻¹¹ M.

Furthermore, the present invention relates to the use of an oligo- or polynucleotide for the detection of a polynucleotide of the invention and/or for genotyping of corresponding individual CYP2B6 alleles. Preferably, said oligo- or polynucleotide is a polynucleotide or a nucleic acid molecule of the invention described before.

In a particular preferred embodiment said oligonucleotide is about 15 to 50, preferably 20 to 40, more preferably 20 to 30 nucleotides in length and comprises the nucleotide sequence of any one of SEQ ID NOs: 1 to 43 or a complementary sequence.

Hence, in a still further embodiment, the present invention relates to a primer or probe consisting of an oligonucleotide as defined above. In this context, the term “consisting of” means that the nucleotide sequence described above and employed for the primer or probe of the invention does not have any further nucleotide sequences of the CYP2B6 gene immediately adjacent at its 5′ and/or 3′ end. However, other moieties such as labels, e.g., biotin molecules, histidin flags, antibody fragments, colloidal gold, etc. as well as nucleotide sequences which do not correspond to the CYP2B6 gene may be present in the primer and probes of the present invention. Furthermore, it is also possible to use the above described particular nucleotide sequences and to combine them with other nucleotide sequences derived from the CYP2B6 gene wherein these additional nucleotide sequences are interspersed with moieties other than nucleic acids or wherein the nucleic acid does not correspond to nucleotide sequences of the CYP2B6 gene. Furthermore, it is evident to the person skilled in the art that the oligonucleotide can be modified, for example, by thio-phosphate-backbones and/or base analogs well known in the art (Flanagan, Proc. Natl. Acad. Sci. USA 96 (1999), 3513-8; Witters, Breast Cancer Res. Treat. 53 (1999), 41-50; Hawley, Antisense Nucleic Acid Drug Dev. 9 (1999), 61-9; Peng Ho, Brain Res. Mol. Brain Res. 62 (1998), 1-11; Spiller, Antisense Nucleic Acid Drug Dev. 8 (1998), 281-93; Zhang, J. Pharmacol. Exp. Ther. 278 (1996), 971-9; Shoji, Antimicrob. Agents Chemother. 40 (1996), 1670-5; Crooke, J. Pharmacol. Exp. Ther. 277 (1996), 923-37).

In addition, the present invention relates to the use of an antibody or a substance capable of binding specifically to the gene product of a CYP2B6 gene for the detection of the variant CYP2B6 protein of the invention, the expression of a molecular variant CYP2B6 gene comprising a polynucleotide of the invention and/or for distinguishing CYP2B6 alleles comprising a polynucleotide of the invention.

Moreover, the present invention relates to a composition, preferably pharmaceutical composition comprising the antibody, the nucleic acid molecule, the vector or the inhibitor of the present invention, and optionally a pharmaceutically acceptable carrier. These pharmaceutical compositions comprising, e.g., the inhibitor or pharmaceutically acceptable salts thereof may conveniently be administered by any of the routes conventionally used for drug administration, for instance, orally, topically, parenterally or by inhalation. Acceptable salts comprise acetate, methylester, HCl, sulfate, chloride and the like. The compounds may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

Furthermore, the use of pharmaceutical compositions which comprise antisense-oligonucleotides which specifically hybridize to RNA encoding mutated versions of a CYP2B6 gene according to the invention or which comprise antibodies specifically recognizing mutated CYP2B6 protein but not or not substantially the functional wild-type form is conceivable in cases in which the concentration of the mutated form in the cells should be reduced.

Thanks to the present invention the particular drug selection, dosage regimen and corresponding patients to be treated can be determined in accordance with the present invention. The dosing recommendations will be indicated in product labeling by allowing the prescriber to anticipate dose adjustments depending on the considered patient group, with information that avoids prescribing the wrong drug to the wrong patients at the wrong dose.

Furthermore, the present invention relates to a diagnostic composition or kit comprising any one of the afore-described polynucleotides, oligonucleotides, probes, vectors, host cells, variant CYP2B6 proteins, antibodies, inhibitors, nucleic acid molecules or the corresponding vectors of the invention, and optionally suitable means for detection.

The kit of the invention may contain further ingredients such as selection markers and components for selective media suitable for the generation of transgenic cells and animals. The kit of the invention may advantageously be used for carrying out a method of the invention and could be, inter alia, employed in a variety of applications, e.g., in the diagnostic field or as research tool. The parts of the kit of the invention can be packaged individually in vials or in combination in containers or multicontainer units. Manufacture of the kit follows preferably standard procedures which are known to the person skilled in the art. The kit or diagnostic compositions may be used for methods for detecting expression of a mutant form of CYP2B6 gene in accordance with any one of the above-described methods of the invention, employing, for example, immunoassay techniques such as radioimmunoassay or enzymeimmunoassay or preferably nucleic acid hybridization and/or amplification techniques such as those described herein before and in the examples.

Some genetic changes lead to altered protein conformational states. For example, some variant CYP2B6 proteins may possess a tertiary structure that renders them far less capable of facilitating drug metabolization and transcription initiation, respectively. Restoring the normal or regulated conformation of mutated proteins is the most elegant and specific means to correct these molecular defects, although it is difficult. Pharmacological manipulations thus may aim at restoration of wild-type conformation of the protein. Thus, the polynucleotides and encoded proteins of the present invention may also be used to design and/or identify molecules which are capable of activating the wild-type function of a CYP2B6 gene or protein.

In another embodiment the present invention relates to the use of a drug or prodrug for the preparation of a pharmaceutical composition for the treatment or prevention of a disorder diagnosed by the method described hereinbefore. Furthermore, the present invention relates to the use of an effective dose of a nucleic acid sequence encoding a functional and expressible wild type CYP2B6 protein for the preparation of a pharmaceutical composition for treating, preventing and/or delaying a disorder diagnosed by the method of the invention. A gene encoding a functional and expressible CYP2B6 protein can be introduced into the cells which in turn produce the protein of interest. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716, WO94/29469; WO 97/00957 or Schaper, Current Opinion in Biotechnology 7 (1996), 635-640, and references cited therein. The gene may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell.

As is evident from the above, it is preferred that in the use of the invention the nucleic acid sequence is operatively linked to regulatory elements allowing for the expression and/or targeting of the CYP2B6 protein to specific cells. Suitable gene delivery systems that can be employed in accordance with the invention may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Standard methods for transfecting cells with recombinant DNA are well known to those skilled in the art of molecular biology, see, e.g., WO 94/29469; see also supra. Gene therapy may be carried out by directly administering the recombinant DNA molecule or vector of the invention to a patient or by transfecting cells with the polynucleotide or vector of the invention ex vivo and infusing the transfected cells into the patient.

In a preferred embodiment of the uses and methods of the invention, said disorder is cancer of the lung, breast and kidney, respectively.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nim.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The pharmaceutical and diagnostic compositions, uses, methods of the invention can be used for the diagnosis and treatment of all kinds of diseases hitherto unknown as being related to or dependent on variant CYP2B6 genes. The compositions, methods and uses of the present invention may be desirably employed in humans, although animal treatment is also encompassed by the methods and uses described herein.

### BRIEF DESCRIPTION OF THE DRAWINGS

FIG. 1: Allelic variants of the human CYP2B6 gene. Exons containing mutations resulting in amino acid changes are shown as black boxes, exons resulting in the wt amino acid sequence are shown in white. Base numbering and allele designation was done in accordance with published recommendations for human allele nomenclature (Antonarakis, 1998; http://www.imm.ki.se/CYPalleles/criteria.htm). Only alleles for which definitive haplotype structures could be obtained are shown.

FIG. 2: Primers and novel polymorphisms in the 5′-flanking sequence of CYP2B6 (Sequence Genbank Accession Number AC023172).

FIG. 3: Human CYP2B6 gene structure and PCR strategy. Exons are shown as boxes with exon size in bp and intron size shown in kb [UT=untranslated region). The position and size of the fragments amplified by PCR are shown underneath. Sequences of primers used for PCR are summarized in Table 1.

FIG. 4: Analysis of CYP2B6 protein expression (A) and enzymatic function (B). CYP2B6 protein was determined by immunoblotting and the S-mephenytoin N-demethylation to Nirvanol was measured in 92 liver microsome samples. The 5 nonsynonymous CYP2B6 mutations were determined in DNA from the same 92 individuals. Wt indicates the group of 30 individuals with homozygous wild-type genotype; 487 R/C and 487 C/C show the results for individuals heterozygous or homozygous for the exon 9 mutation, respectively. All other mutant genotypes are collectively shown. The average expression and activity of each group is shown as horizontal line. Statistical significance was evaluated by the t-test in comparison to the Wt group: * P<0.05; ** P<0.01.

FIG. 5: Analysis of CYP2B6 mRNA expression in human liver. CYP2B6 mRNA was determined by a specific RT-PCR TaqMan assay and measured in 102 liver microsome samples. The 5 nonsynonymous CYP2B6 mutations were determined in DNA from the same 102 individuals. The figure shows the results for the group of 31 individuals homozygous for the wild type allele (WT) and 26 individuals heterozygous for the 172 and the 262 mutations. The means are shown as horizontal lines. The difference between both groups was statistically significant (Mann-Whitney-test: ***P<0.001).

The invention will now be described by reference to the following examples which are merely illustrative and are not to be construed as a limitation of the scope of the present invention.

### EXAMPLES

### DNA Samples and PCR Conditions

Leucocyte DNA was isolated by standard methods from 35 German Caucasian individuals. To ensure amplification of CYP2B6 versus the CYP2B7 pseudogene amplification was done with intron-specific and 5′-upstream-specific PCR primer pairs (Table 1). The PCR reactions were performed in a total volume of 50 μl with 100 ng genomic DNA, 50 mM KCl, 10 mM Tris-HCl pH8.3, 1.5 mM MgCl₂, 0.5% Tween 20, 0.01% Bovine Serum Albumine, 200 μM dNTPs, 2 pMol of each primer and 0.5 U Taq-Polymerase (Promega, Madison). After 5 minutes of denaturation at 95° C., the samples were subjected to the following amplification program: 95° C. for 30 s, 50-60° C for 30 s, and 72° C for 30 s−2 min for 30 cycles with a delayed last step of 10 min at 72° C. Aliquots of each PCR product were subjected to agarose gel electrophoresis to ensure proper amplification. Aliquots of each PCR sample were digested with restriction endonucleases under conditions according to the manufacturers instruction, and the resulting fragments were analyzed on Agarosegel stained with ethidium bromide

### PCR Product Sequencing

PCR products were directly sequenced using infrared-800 labeled nested primers (MWG Biotech, Ebersberg, Germany) with the Thermo Sequenase fluorescent labelled primer cycle sequencing kit (Amersham Life Sciences, Little Chalfont, England). The nucleotide sequences are shown in Table 1. Sequencing analysis was performed on an automated DNA sequencer (Licor 4200, MWG Biotech, Ebersberg, Germany) using Base ImagIR data collection and image analysis 4.00 software.

### Example 1

### Detection of CYP2B6 Mutations by PCR-RFLP

1.1 C64T Mutation in Exon 1

PCR reactions were performed in a total volume of 50 μl with 100 ng genomic DNA, 200 μM dNTPs, 2 pmol of primer CYP2B6-1F and primer CYP2B6-1R (Table1), 10 mM Tris-HCl pH 7.0, 50 mM KCl, 1.5 mM MgCl₂, 0.5% Tween 20, 0.01% bovine serum albumine and 2.5 U Taq DNA polymerase (Promega Corporation, Madison, USA). After 5 min of denaturation at 95° C. the PCR mixtures were subjected to the following conditions : 30 sec 95° C., 30 sec 50° C. and 1 min 72° C. for 30 cycles with a delayed last step for 10 min at 72° C. in a PTC200 thermal cycler (MJ Research Inc. Watertown, Mass.). PCR products were purified with the Quiaquick PCR purification kit (Qiagen, Hilden, Germany). Aliquots of each sample were digested with Hae II (Roche, Basel, Switzerland), and the resulting fragments were analyzed on a 2% Agarosegel (Gibco-BRL, Eggenstein, Germany) stained with ethidium bromide. Hae II digestion of wildtype DNA yields fragments of 333 and 390 bp, whereas DNA containing the C64T mutation is not digested and yields an uncleaved fragment of 723 bp.

1.2 G516T Mutation in Exon 4

For amplification of exon 4, the primer pair CYP2B6-4F/-4R was used with the same cycling and analysis conditions as for exon 1, except that 56° C. was used as annealing temperature and the extension step was reduced to 40 seconds. The purified PCR products were digested with restriction enzyme BsrI (New England Biolabs, Beverly, USA) for 1 hour at 60° C. Wild-type DNA resulted in three fragments of 241 bp, 268 bp and 17 bp, whereas mutant PCR products resulted in two fragments of 509 bp and 17 bp.

1.3 C777A Mutation in Exon5

Exon 5 was amplified with primers CYP2B6-5F and CYP2B6-5R again with the same cycling conditions as for exon 1 besides that the annealing temperature was 60° C. The mutation could be detected by digestion with restriction enzyme Hae II leaving mutated DNA at postion 777 undigested. The wildtype PCR fragments are 140, 196 and 304 bp, whereas in mutated samples an additional fragment of 500 bp could be detected.

1.4 A785G Mutation in Exon 5

The same PCR fragment as for the C777A mutation could be analyzed for another mutation in exon 5. The A785G mutation destroys a recognition sequence for the restricition enzyme Sty I (Roche, Basel, Switzerland). Digestion of wildtype DNA results in fragments of 56, 116, 171 and 297 bp. The A785G mutation creates a fragment of 468 bp. The fragments were analyzed in a 2.5% MetaPhor Agarosegel (FMC, Rockland, USA) where fragments 56 and 116 could not be resolved.

1.5 C1459T Mutation in Exon 9

Exon 9 was amplified with primers CYP2B6-9F and primer CYP2B6-9R using the same conditions as for exon 1 with the exception of a prolonged extension step of 1 min 30 sec and a raised annealing step at 60° C. The C1459T mutation introduces a restriction enzyme site for Bgl II (Roche, Basel, Switzerland) which yields fragments of 216 and 1185 bp. Wildtype DNA yields an uncleaved PCR fragment of 1401 bp. The fragments were separated in a 1.5% Agarosegel and could be detected after ethidium bromide staining.

### Example 2

### Polymorphisms in the CYP2B6 5′-flanking Sequence (PCR-RFLP) and Functional Analysis of CYP2B6 Polymorphisms in a Human Liver Bank

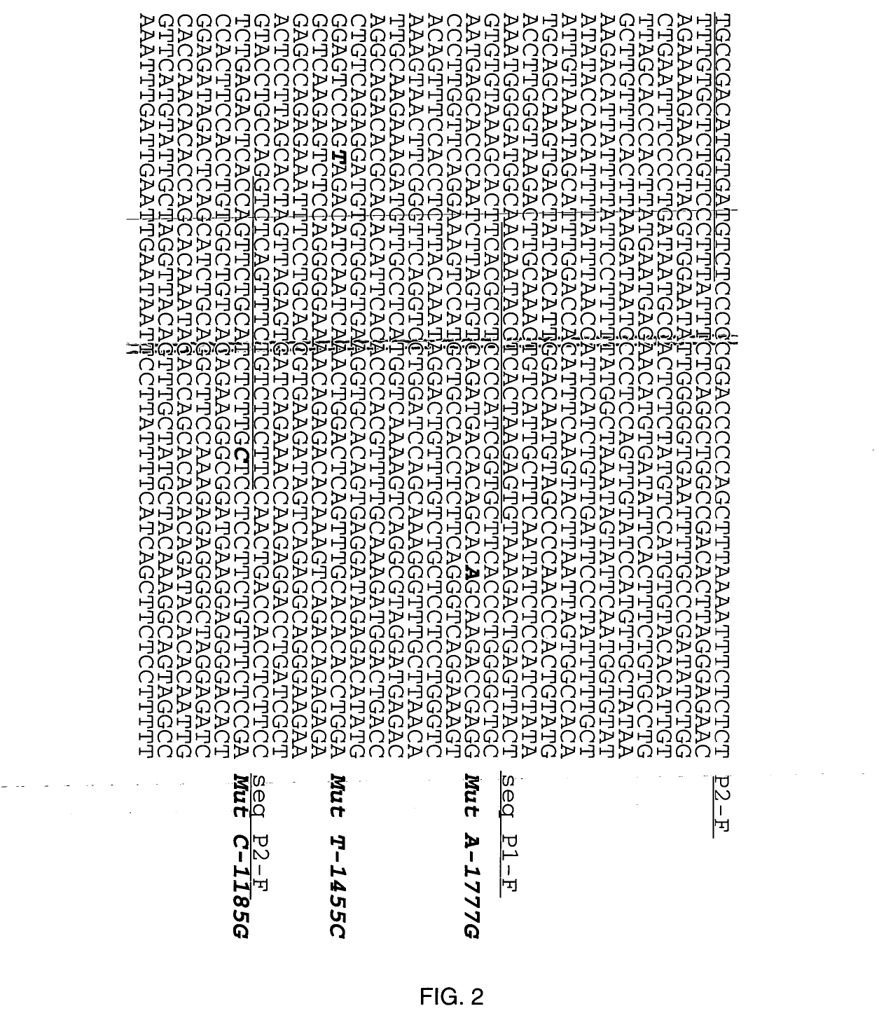

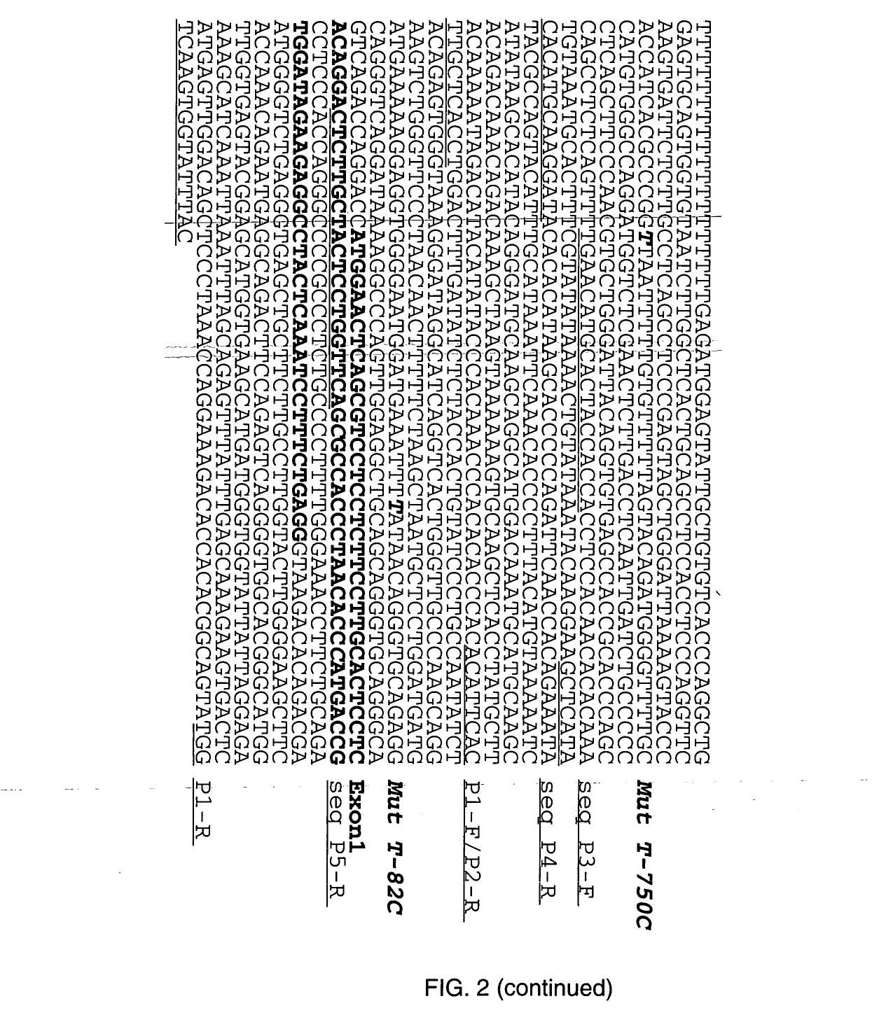

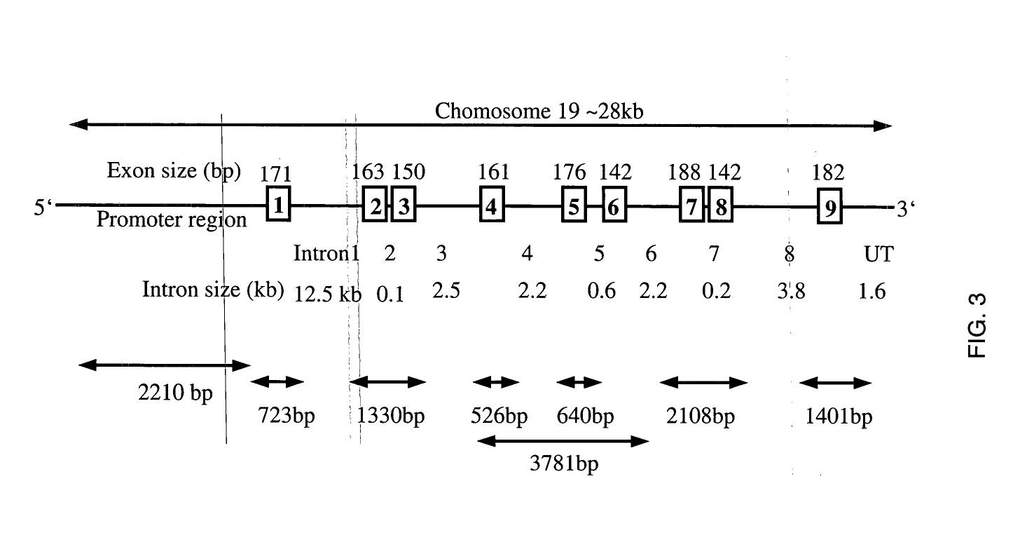

A specific direct DNA sequencing strategy was developed to amplify 2.5 kb of the CYP2B6 5′-flanking region. FIG. 2 shows the 5′-flanking sequence and the location of the primers used for its specific amplification and sequencing (see also Table 1). Five novel mutations were identified by complete sequence analysis of DNA from 35 individuals. These are: T-82C, T-750C, C-1185G, T-1455C, A-1777G (Table 2). The frequencies of these mutations varied between 1.4 and 54.2% (Table 2).

### Example 3

### Description of the Results

Genomic sequences of the human CYP2B locus were used which were elucidated in the course of the human genome project and made publically available through the internet http://www-bio.llnl.gov/genome/genome.html (Annotation: Click on “Genomic sequencing of regions of chr 19”, and enter the clone id22376 in the query box at the bottom of the form. This take you to the tiling path, with the list (ordered from centromere to telomere) of clones sequenced, and links to their sequence and accessions (when available) in Genbank. The cosmid F22376 spans the entire CYP2B6 gene and is currently sequenced but is still in the annotation stage.) to design oligonucleotide primers to amplify genomic DNA containing the CYP2B6 coding regions (Table 1). It was shown by sequence comparison that the amplified segments had been specifically derived from the CYP2B6 gene and not from CYP2B7 in all cases.

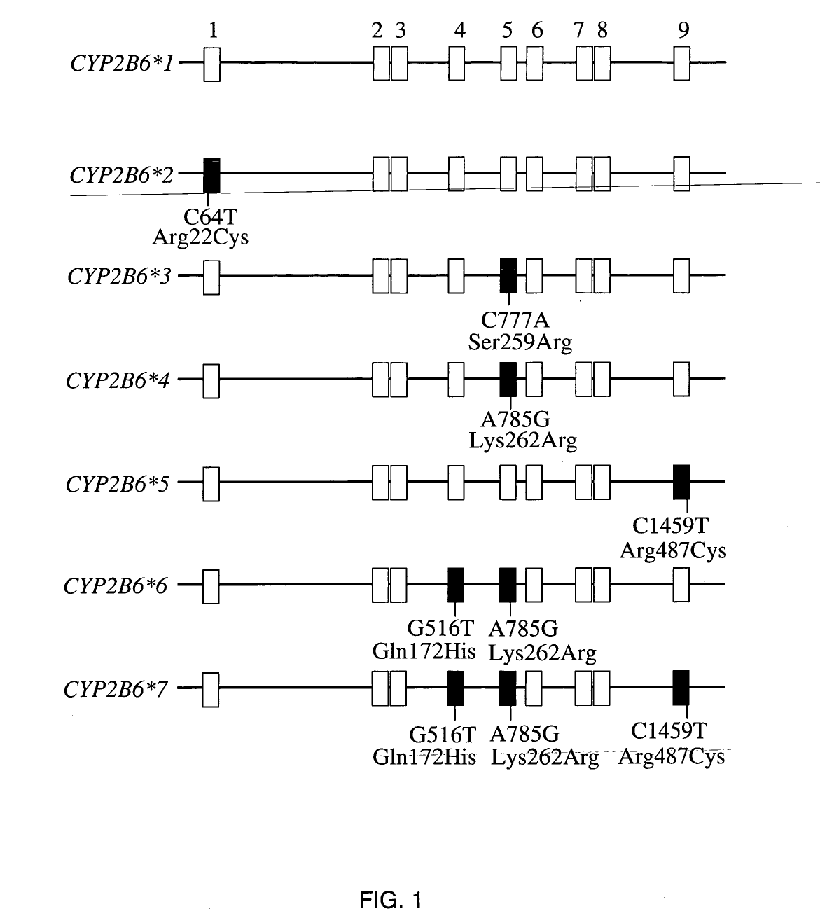

To search for polymorphism, genomic DNA from 35 randomly selected, healthy Caucasian individuals was amplified with all CYP2B6-specific primer pairs and the amplified fragments were completely sequenced in both directions. A total of five non-synonymous mutations were detected in exons 1, 4, 5 and 9, which occured with different frequencies and in different heterozygous and homozygous combinations (Table 2). Linkage analysis resulted in the elucidation of 7 different alleles summarized in Table 3 and FIG. 1. Analysis of the data obtained from the 35 completely sequenced individuals revealed a total of 6 mutant alleles in addition to the wild-type allel (Table 3).

Diagnostic tests were developed to easily detect each of the five mutations in genomic DNA. Each Mutation could be shown to either abolish or create an enzymatic restriction site (Table 2). It was thus possible in all cases to develop an assay based on 1) CYP2B6-specific amplification of the gene fragment that contains the mutation and 2) digestion with a suitable restriction enzyme. The frequency of each mutation was estimated in a representative German population using the dignostic test designed for it (Table 2).

### Example 4

### Analysis of the Functional Significance of the CYP2B6 Mutations

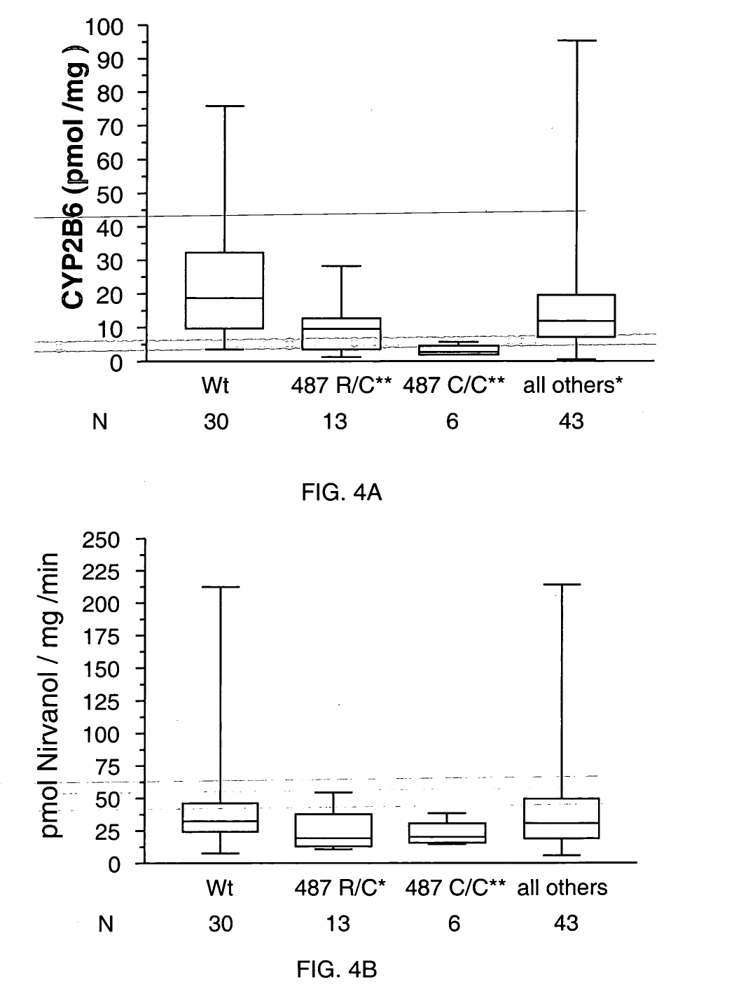

The functional significance of the mutations in the CYP2B6 gene (Table 2) was investigated by analyzing expression and enzymatic activity of CYP2B6 in a large human liver bank. A specific monoclonal antibody, MAB-2B6 (Gentest Corp., Woburn, Mass., USA) was used to immunoquantitate microsomal CYP2B6. S-mephenytoin N-demethylation to Nirvanol was measured as a rather CYP2B6-specific pathway. The five nonsynonymous mutations were determined in all samples. Compared to the homozygous wild-types, who expressed on average 23.5±16.3 pmol/mg CYP2B6, significantly reduced levels were measured for carriers of the C1459T (R487C) mutation (alleles *5 and *7): heterozygotes expressed 9.7±7.5 pmol/mg (P=0.006, N=13) and homozygotes only 3.05±1.4 pmol/mg (P=0.005, N=6, t-test; FIG. 4A). Thus, compared to the homozygous wild-type, CYP2B6 protein expression of homozygous carriers of the R487C mutation in exon 9 was almost eightfold reduced. The mean enzyme activities of heterozygous (25.9±15.2 pmoles of nirvanol formed/min*mg, P=0.02, t-test) and homozygous carriers of R487C (22.2±8.4, P=0.008, t-test) were also significantly reduced compared to the Wt group (49.2±49.4; FIG. 4B).

A significant difference in protein expression was also found for individuals with other mutation than the exon 9 mutation, who expressed 16.9±18 pmol/mg, which was significantly less compared to the homozygous wild-type (P=0.016; FIG. 4A).

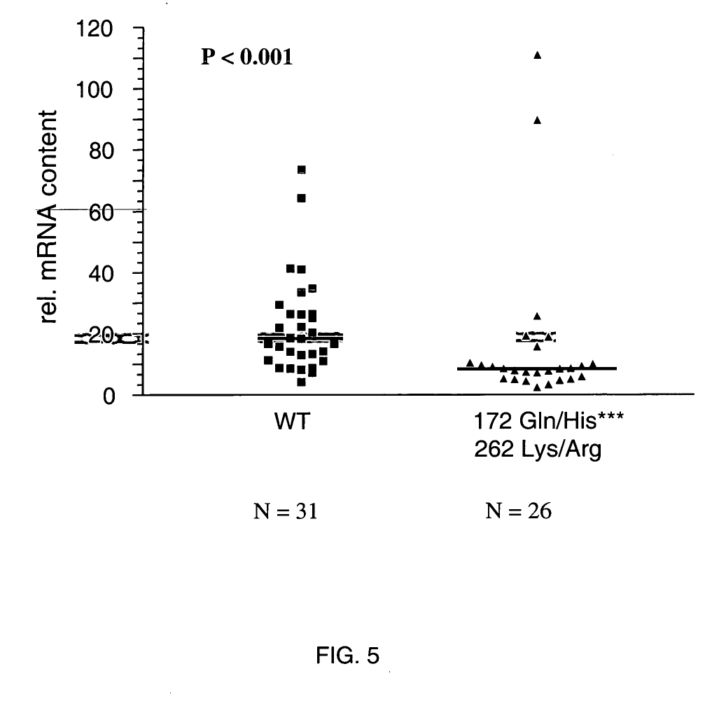

Finally, quantitation of CYP2B6 mRNA transcripts was performed using a newly developed real-time PCR assay in total RNA prepared from human livers. As shown in FIG. 5, there was a significant difference in mRNA expression between the homozygous wild-types and the group of individuals being heterozygous for the 172 Gln>His and the 262 Lys>Arg mutations, who expressed only about 50% of the transcripts (P<0.001). The decrease in mRNA expression is also influenced by linkage to the promoter mutation T-750C, which is present at about 54% and which affects a putative hepatic nuclear factor 1 (hNF1) consensus binding site.

### Example 5

### Significance of the Results

Genetic polymorphisms in drug metabolizing enzymes have been shown to be one of the major factors of interindividual variations in drug response. Genetic polymorphism may affect gene expression, mRNA and protein stability, enzyme activity and/or substrate specificity for a given gene product and thus leads to complex variations in the way the organism acts on the drug. The precise consequences a mutation may have depends on the drug in question, e.g. whether it is a prodrug or not, or whether the metabolites formed are themselves active or toxic.

A number of clinically important drugs have been shown to be substrates for CYP2B6, including the anticancer prodrugs cyclophosphamide and ifosfamide, the antioestrogenic tamoxifen, the platelet aggregation inactivator, clopidogrel, but also addictive drugs including nicotine and ecstasy (Table 4).

CYP2B6 is involved in the metabolic activation and inactivation of a number of clinically important drugs such as the frequently used antineoplastic agents cyclophosphamide and ifosfamide, the antioestrogen tamoxifen, the newer platelet aggregation inhibitor Clopidogrel, but also environmental toxicants and recreational drugs such as nicotine and ecstasy (Table 4).

Through genomic sequence analysis, evidence was obtained for a total of 7 distinct alleles which contain between one and three amino acid mutations. Some of these alleles occur with high frequency in the normal population. The biochemical properties of the CYP2B6 protein such as protein stability, activity, or substrate specificity are changed by these mutations and will lead to interindividual differences in drug metabolism.

The use of diagnostic tests for the newly identified mutations will help to predict therapeutic efficacy and/or drug toxicity related to drugs metabolized by CYP2B6.

In addition, since CYP2B6 was also shown to participate in nicotine metabolism, CYP2B6 genetic diagnosis will help to predict smoking habits (Pianezza, Nature 393 (1998), 750).

TABLE 1A

Sequences of primers used for PCR
amplification of CYP2B6 coding exons

| Primer | | Primer | Amplified | Product |
|---|---|---|---|---|
| designation | 5′-3′ sequence | location | exon | size (bp) |
| CYP2B6-1F | ACATTCACTTGCTCACCT | 5′ UT | 1 | 723 |
| CYP2B6-1R | GTAAATACCACTTGACCA | Intron 1 | | |
| CYP2B6-2F | ATCCTACTCAGAATGATGCACAAC | Intron 1 | 2 + 3 | 1330 |
| CYP2B6-3R | ATTACAGGTGAGAGTCATCACATC | Intron 3 | | |
| CYP2B6-4F | GGTCTGCCCATCTATAAAC | Intron 3 | 4 | 526 |
| CYP2B6-4R | CTGATTCTTCACATGTCTGCG | Intron 4 | | |
| CYP2B6-4F | TCCCTGGGATTTAACTGTACTCAC | Intron 3 | 4 + 5 + 6 | 3781 |
| 1 | | | | |
| CYP2B6-6R | CAGAATTGGCTTGGTTGGAATCTA | Intron 7 | | |
| CYP2B6-5F | GACAGAAGGATGAGGGAGGAA | Intron 4 | 5 | 640 |
| CYP2B6-5R | CTCCCTCTGTCTTTCATTCTGT | Intron 5 | | |
| CYP2B6-7F | GTGATTATTCATTAATTGGGTTC | Intron 6 | 7 + 8 | 2108 |
| CYP2B6-8R | TGCAATGGTTGATTGATGCTC | Intron 8 | | |
| CYP2B6-9F | TGAGAATCAGTGGAAGCCATAGA | Intron 8 | 9 | 1401 |
| CYP2B6-9R | TAATTTTCGATAATCTCACTCCTG | 3′ UT | | |
| | C | | | |

TABLE 1B

Sequences of primers used for PCR
amplification of CYP2B6 promoter region

| Primer | | Primer | Product |
|---|---|---|---|
| designation | 5′-3′ sequence | location | size (bp) |
| CYP2B6-P1F | ACATTCACTTGCTCACCT | 5′ upstream | 723 |
| CYP2B6-P1R | GTAAATACCACTTGACCA | Intron 1 | |
| CYP2B6-P2F | TGCCGACATGTGATGTCT | 5′ upstream | 2210 |
| CYP2B6-P2R | AGGTGAGCAAGTGAATGT | 5′ upstream | |

TABLE 1C

Sequences of primers used for
sequencing of CYP2B6 coding exons

| | | | |
|---|---|---|---|
| Sequence | | | |
| primer | | Primer | |
| designation | 5′-3′sequence | location | Exon |

| | | | |
|---|---|---|---|
| seqCYP2B6-1F | ATAACAGGGTGCAGAGGCA | 5′ UT | 1 |
| seqCYP2B6-1R | AAGTACCAAGGCAAGAAGCA | Intron 1 | 1 |
| seqCYP2B6-2F | GGCTAATTACCAATCTGGT | Intron 1 | 2 |
| seqCYP2B6-2R | ATATACTCCCTTCCCTGATGCA | Intron 2 | 2 |
| seqCYP2B6-3F | ACTCAGAGCCTTCTTCCAACT | Intron 2 | 3 |
| seqCYP2B6-3R | ACCTGCATCTCTCAGTGTTTCATT | Intron 3 | 3 |
| seqCYP2B6-4F | TAGGTGACAGCCTGATGTTC | Intron 3 | 4 |
| seqCYP2B6-4R | TCATCCTTTTCTCGTGTGTTCT | Intron 4 | 4 |
| seqCYP2B6-5F | TCTCTCCCTGTGACCTGCTAGCT | Intron 4 | 5 |
| seqCYP2B6-5R | TCTTCTCACCTCTCCATCTT | Intron 5 | 5 |
| seqCYP2B6-6F | TATACACAGCAAGGCTACAG | Intron 5 | 6 |
| seqCYP2B6-6R | ATTTCTGCAGCTCAGAAGGA | Intron 6 | 6 |
| seqCYP2B6-7F | GATTACAGGCATGAGCCACCAT | Intron 6 | 7 |
| seqCYP2B6-7R | ATTAAGAGAATCCAGGATGCC | Intron 7 | 7 |
| seqCYP2B6-8F | TTTTGTGGAGTGTGTGGAGGGT | Intron 7 | 8 |
| seqCYP2B6-8R | TTCTCAAGTTGGGGATAGTA | Intron 8 | 8 |
| seqCYP2B6-9F | AGAGCGAAGTGTATGCACCT | Intron 8 | 9 |
| seqCYP2B6-9R | AGAGCCATTGTCTACAGAGG | 3′ UT | 9 |

TABLE 1D

Sequences of primers used for
sequencing CYP2B6 promoter region

| Sequence primer | | Primer |
|---|---|---|
| designation | 5′-3′sequence | location |
| seqCYP2B6-P1F | ACAATACCTCACTAAGAGTG | 5′ upstream |
| seqCYP2B6-P2F | GTCTCAGTTTCTGTCTCCTTC | 5′ upstream |
| seqCYP2B6-P3F | TGAACATGCACTACCACCA | 5′ upstream |
| seqCYP2B6-P4R | ATCCTTGCATGTGTATGAGC | 5′ upstream |
| seqCYP2B6-P5R | TGAACCAGGAGTAGCAAGAG | Exon 1 |

TABLE 2
Total frequencies of CYP2B6 mutations^{a}

| Nucleotide | Predicted | | | Frequency |
|---|---|---|---|---|
| change | consequence | Location | RFLP | (%) |
| A-1777G | unknown | 5′ upstream | BceF I created | 8.6 |
| T-1455C | unknown | 5′ upstream | Acc I abolished | 31.4 |
| C-1185G | unknown | 5′ upstream | Ava II created | 5.7 |
| T-750C | unknown | 5′ upstream | Mse I abolished | 54.2 |
| T-82C | unknown | 5′ upstream | no sites found | 1.4 |
| C64T | Arg22Cys | Exon 1 | Hae II abolished | 5.3 |
| G516T | Gln172His | Exon 4 | Bsr I abolished | 28.6 |
| C777A | Ser259Arg | Exon 5 | Hae II abolished | 0.5 |
| A785G | Lys262Arg | Exon 5 | Sty I abolished | 32.6 |
| C1459T | Arg487Cys | Exon 9 | Bgl II created | 14.0 |
| C78T | silent | Exon 1 | BspH I created | 2.9 |
| G216C | silent | Exon 2 | EcoR II created | 7.1 |
| G714A | silent | Exon 5 | Pst I abolished | 1.4 |
| C732T | silent | Exon 5 | no sites found | 2.9 |
| A59T | unknown | Intron 2 | — | 7.1 |
| C-18T | unknown | Intron 3 | — | 21.4 |

^{a}Frequencies were determined in 215 individuals by using PCR-RFLP tests for nonsynonymous mutations and by sequencing in 35 individuals.

TABLE 3
Alleles of CYP2B6

| Allele | Nucleotide changes | Amino acid mutations |
|---|---|---|
| CYP2B6*1 | none | |
| CYP2B6*2 | C64T | Arg22Cys |
| CYP2B6*3 | C777A | Ser259Arg |
| CYP2B6*4 | A785G | Lys262Arg |
| CYP2B6*5 | C1459T | Arg487Cys |
| CYP2B6*6 | G516T; A785G | Gln172His; Lys262Arg |
| CYP2B6*7 | G516T; A785G; C1459T | Gln172His; Lys262Arg; |
| | | Arg487Cys |

TABLE 4
Substrates for CYP2B6

| Substrate | Type | Reaction catalyzed | Reference |
|---|---|---|---|
| Cyclo- | Anti-cancer | Aliphatic | Chang et al |
| phosphamide | drug | hydroxylation, | 1993 |
| | | N-dealkylation | |
| Iphosphamide | Anti-cancer | Aliphatic | Granvil et al., |
| | drug | hydroxylation, | 1999 |
| | | N-dealkylation | |
| S-Mephenytoin | Anticonvulsant | N-Demethylation | Heyn et al., |
| | | | 1996 |
| Tamoxifen | Anti-oestrogen | Hydroxylation | Gillam et al., |
| | | | 1999 |
| RP 73401 | | | Stevens et |
| | | | al., 1997 |
| Clopidogrel | Platelet | Hydroxylation | Coukell and |
| | aggregation | | Markham, |
| | inhibitor | | 1997 |
| Nicotine | Major tobacco | C-oxidation | Flammang et |
| | constituent | | al., 1992 |
| MDMA | recreational | N-dealkylation | |
| (“ecstasy”) and | drugs | | |
| MDEA (“eve”) | | | |
| Dibenzo-[a,h]- | Precarcinogen | | Shou et al., |
| anthracene | | | 1996 |
| 6- | Precarcinogen | | Mimura et |
| Aminochrysene | | | al., 1993 |

### References

Ariyoshi N, Oguri K, Koga N, Yoshimura H, Funae Y. Metabolism of highly persistent PCB congener, 2,4,5,2′,4′,5′-hexachlorobiphenyl, by human CYP2B6. Biochem Biophys Res Commun 1995 Jul. 17; 212(2): 455-60

Carrano A V, Lamerdin J, Ashworth L K, Watkins B, Branscomb E, Slezak T, Raff M, de Jong P J, Keith D, McBride L, et al. A high-resolution, fluorescence-based, semiautomated method for DNA fingerprinting. Genomics 1989 February; 4(2): 129-36

Chang T K, Weber G F, Crespi C L, Waxman D J. Differential activation of cyclophosphamide and ifosphamide by cytochromes P-450 2B and 3A in human liver microsomes. Cancer Res 1993 Dec. 1; 53(23): 5629-37

Coukell A J, Markham A., Clopidogrel. Drugs 1997: 54, 745 -750

Gervot L, Rochat B, Gautier J C, Bohnenstengel F, Kroemer H, de Berardinis V, Martin H, Beaune P, de Waziers I. Human CYP2B6: expression, inducibility and catalytic activities. Pharmacogenetics 1999 June; 9(3): 295-306

Code E L, Crespi C L, Penman B W, Gonzalez F J, Chang T K, Waxman D J. Human cytochrome P4502B6: interindividual hepatic expression, substrate specificity, and role in procarcinogen activation. Drug Metab Dispos 1997 August; 25(8): 985-93

Gonzalez F J, Crespi C L, Czerwinski M, Gelboin H V. Analysis of human cytochrome P450 catalytic activities and expression. Tohoku J Exp Med 1992 October; 168(2): 67-72

Granvil C P, Madan A, Sharkawi M, Parkinson A, Wainer I W. Role of CYP2B6 and CYP3A4 in the in vitro N-dechloroethylation of (R)- and (S)-ifosfamide in human liver microsomes. Drug Metab Dispos 1999 April; 27(4): 533-41

Hoffman S M, Fernandez-Salguero P, Gonzalez F J, Mohrenweiser H W. Organization and evolution of the cytochrome P450 CYP2A-2B-2F subfamily gene cluster on human chromosome 19. J Mol Evol 1995 December; 41(6): 894-900

Kobayashi K, Abe S, Nakajima M, Shimada N, Tani M, Chiba K, Yamamoto T. Role of human CYP2B6 in S-mephobarbital N-demethylation. Drug Metab Dispos 1999 December; 27(12): 1429-33

Klaus-Peter Kreth, Karl-Artur Kovar, Matthias Schwab, Ulrich M. Zanger. Identification of the Human Cytochromes P450 involved in the Oxidative metabolism of “Ecstasy”-Related Designer-Drugs. Biochemical Pharmacology 2000 (in press)

Miles J. S., McLaren A. W., Wolf C. R. A novel human cytochrome P450 gene (P450IIB): chromosomal localization and evidence for alternative splicing. Nucleic Acids Res. 16:5783-5795 (1987).

Miles, J. S., Spurr, N. K., Gough, A. C., Jowett, T., McLaren, A. W., Brook, J. D. and Wolf, C. R. A novel human cytochrome P450 gene (P450IIB): chromosomal localization and evidence for alternative splicing. Nucleic Acids Res. 16 (13), 5783-5795 (1988)

Miles J. S., McLaren A. W., Wolf C. R. Alternative splicing in the human cytochrome P45011B6 gene generates a high level of aberrant messages. Nucleic Acids Res. 17: 8241-8255 (1989).

Mimura M, Baba T, Yamazaki H, Ohmori S, Inui Y, Gonzalez F J, Guengerich F P, Shimada T. Characterization of cytochrome P-450 2B6 in human liver microsomes. Drug Metab Dispos 1993 November-December; 21(6): 1048-56

Shou M, Krausz K W, Gonzalez F J, Gelboin H V. Metabolic activation of the potent carcinogen dibenzo[a,h]anthracene by cDNA-expressed human cytochromes P450. Arch Biochem Biophys 1996 April 1;328(1):201-7

Stevens J C, White R B, Hsu S H, Martinet M. Human liver CYP2B6-catalyzed hydroxylation of RP 73401. J Pharmacol Exp Ther 1997 September; 282(3): 1389-95

Yamano, S., Nhamburo, P. T., Aoyama, T., Meyer, U. A., Inaba, T., Kalow, W., Gelboin, H. V., McBride, O. W. and Gonzalez, F. J. cDNA cloning and sequence and cDNA-directed expression of human P450 IIB1: identification of a normal and two variant cDNAs derived from the CYP2B locus on chromosome 19 and differential expression of the IIB mRNAs in human liver. Biochemistry 28 (18), 7340-7348 (1989)

Ekins, S., VandenBranden M, Ring, B. J., Wrighton, S. A., Eamination of puported probes of human CYP2B6, Pharmacogenetics 1997 June:7(3): 165-79

Heyn H., White, R. B., Stevens, J. C., Catalytic role of cytochrome P4502B6 in the N-demethylation of S-mephnytoin Drug Metab. Dispos. 24(9), 948-954 (1996)

Kobayashi K., Abe, S., Nakajima, M., Shimada, N., Tani, M., Chiba, K., Yamamoto Role of human CYP2B6 in S-mephobarbital N-demethylation. Drug Metab. Dispos. 27(12), 1429-1433 (1999)

Kumar, G. N., Dykstra, J., Roberts, E. M., Jayanti, V. K., Hickman, D., Uchic, J., Yao, Y., Surber B., Thomas, S., Granneman, G. R., Potent inhibition of the cytochrome P-450 3A-mediated human liver microsomal metabolism of a novel HIV protease inibitor by ritonavir: A positive drug-drug interaction, Drug Metab. Dispos. 27(8), 902-908 (1999)

Yanev, S., Kent, U. M., Pandova, B., Hollenberg, P. F., Selective mechanism-based inactivation of cytochromes P-450 2B1 and P-450 2B6 by a series of xanthates, Drug Metab. Dispos. 27(5), 600-604 (1999)

1. A polynucleotide comprising a polynucleotide selected from the group consisting of:
(a) a polynucleotide having the nucleic acid sequence of any one of SEQ ID NO: 44 to 59;
(b) a polynucleotide encoding a polypeptide having the amino acid sequence of any one of SEQ ID NO: 60 to 64;
(c) a polynucleotide capable of hybridizing to a molecular variant of the cytochrome P450 (CYP)2B6 gene, wherein said polynucleotide is having at a position corresponding to position −1777, −1455, −1185, −750 or −82 of the CYP2B6 gene (accession No: AC023172, wherein nucleotide C at position 4115 has been numbered −1), at a position corresponding to position −18 of the CYP2B6 gene (accession No: AC023172, wherein nucleotide G at position 19714 has been numbered −1), at a position corresponding to position 59 of the CYP2B6 gene (accession No: AC023172, wherein nucleotide G at position 16974 has been numbered +1), or at a position corresponding to position 64, 78, 216, 516, 714, 732, 777, 785, or 1459 of the CYP2B6 gene (accession No: M29874, wherein nucleotide A at position 7 has been numbered +1), at least one nucleotide substitution, deletion and/or addition;
(d) a polynucleotide capable of hybridizing to a molecular variant of the cytochrome P450 (CYP)2B6 protein, wherein said polynucleotide is having at a position corresponding to position −18 of the CYP2B6 gene (accession No: AC023172, wherein nucleotide G at position 19714 has been numbered −1), at a position corresponding to position 59 of the CYP2B6 gene (accession No: AC023172, wherein nucleotide G at position 16974 has been numbered +1) or at a position corresponding to position 64, 78, 516, 732, or 1459 of the CYP2B6 gene (accession No: M29874, wherein nucleotide A at position 7 has been numbered +1) a T, at a position corresponding to position 785 of the CYP2B6 gene (accession No: M29874, wherein nucleotide A at position 7 has been numbered +1) or at a position corresponding to position −1185 or −1777 of the CYP2B6 gene (accession No: AC023172, wherein nucleotide C at position 4115 has been numbered −1) a G, at a position corresponding to position −1455, −750, −82 of the CYP2B6 gene (accession No: AC023172, wherein nucleotide C at position 4115 has been numbered −1) or at a position corresponding to position 216 of the CYP2B6 gene (accession No: M29874, wherein nucleotide A at position 7 has been numbered +1) a C or at a position corresponding to position 714 or 777 of the CYP2B6 gene (accession No: M29874, wherein nucleotide A at position 7 has been numbered +1) an A;
(e) a polynucleotide encoding a CYP2B6 polypeptide or fragment thereof, wherein said polypeptide comprises at least one amino acid deletion, addition and/or substitution at an amino acid position corresponding to amino acid residue Arg22 in exon 1, Gln172 in exon 4, Ser259 and/or Lys262 in exon 5 and/or Arg487 in exon 9 of the CYP2B6 polypeptide (accession No: M29874);
(f) a polynucleotide encoding a CYP2B6 polypeptide or fragment thereof, wherein said polypeptide comprises an amino acid substitution of Arg22 to Cys in exon 1, Gln172 to His in exon 4, Ser259 to Arg and Lys262 to Arg in exon 5 and/or Arg487 to Cys in exon 9 of the CYP2B6 polypeptide (accession No: M29874);
(g) a polynucleotide comprising a nucleotide sequence which is not cleaved by the restriction endonuclease HaeII and which is obtainable by PCR amplification from human genomic DNA using oligonucleotides having the SEQ ID NO: 1 and 2 as primers, wherein said polynucleotide is capable of hybridizing to the CYP2B6 gene;
(h) a polynucleotide comprising a nucleotide sequence which is cleaved by the restriction endonuclease BsrI one time and which is obtainable by PCR amplification from human genomic DNA using oligonucleotides having the SEQ ID NO: 5 and 6 as primers, wherein said polynucleotide is capable of hybridizing to the CYP2B6 gene;
(i) a polynucleotide comprising a nucleotide sequence which is cleaved by the restriction endonuclease HaeII one time and which is obtainable by PCR amplification from human genomic DNA using oligonucleotides having the SEQ ID NO: 9 and 10 as primers, wherein said polynucleotide is capable of hybridizing to the CYP2B6 gene;
(j) a polynucleotide comprising a nucleotide sequence which is cleaved by the restriction endonuclease StyI two times and which is obtainable by PCR amplification from human genomic DNA using oligonucleotides having the SEQ ID NO: 9 and 10 as primers, wherein said polynucleotide is capable of hybridizing to the CYP2B6 gene;
(k) a polynucleotide comprising a nucleotide sequence which is cleaved by the restriction endonuclease Bgl II one time and which is obtainable by PCR amplification from human genomic DNA using oligonucleotides having the SEQ ID NO: 13 and 14 as primers, wherein said polynucleotide is capable of hybridizing to the CYP2B6 gene;
(l) a polynucleotide comprising a nucleotide sequence which is cleaved by the restriction endonuclease BspHI one time and which is obtainable by PCR amplification from human genomic DNA using oligonucleotides having the SEQ ID NO: 1 and 2 as primers, wherein said polynucleotide is capable of hybridizing to the CYP2B6 gene;
(m) a polynucleotide comprising a nucleotide sequence which is cleaved by the restriction endonuclease EcoRII nine times and which is obtainable by PCR amplification from human genomic DNA using oligonucleotides having the SEQ ID NO: 2 and 3 as primers, wherein said polynucleotide is capable of hybridizing to the CYP2B6 gene;
(n) a polynucleotide comprising a nucleotide sequence which is not cleaved by the restriction endonuclease PstI and which is obtainable by PCR amplification from human genomic DNA using oligonucleotides having the SEQ ID NO: 9 and 10 as primers, wherein said polynucleotide is capable of hybridizing to the CYP2B6 gene;
(o) a polynucleotide comprising a nucleotide sequence which is cleaved by the restriction endonuclease BceFI two times and which is obtainable by PCR amplification from human genomic DNA using oligonucleotides having the SEQ ID NO: 42 and 43 as primers, wherein said polynucleotide is capable of hybridizing to the CYP2B6 gene;
(p) a polynucleotide comprising a nucleotide sequence which is cleaved by the restriction endonuclease AccI one time and which is obtainable by PCR amplification from human genomic DNA using oligonucleotides having the SEQ ID NO: 42 and 43 as primers, wherein said polynucleotide is capable of hybridizing to the CYP2B6 gene;
(q) a polynucleotide comprising a nucleotide sequence which is cleaved by the restriction endonuclease AvaII six times and which is obtainable by PCR amplification from human genomic DNA using oligonucleotides having the SEQ ID NO: 42 and 43 as primers, wherein said polynucleotide is capable of hybridizing to the CYP2B6 gene; and
(r) a polynucleotide comprising a nucleotide sequence which is cleaved by the restriction endonuclease MseI nine times and which is obtainable by PCR amplification from human genomic DNA using oligonucleotides having the SEQ ID NO: 42 and 43 as primers, wherein said polynucleotide is capable of hybridizing to the CYP2B6 gene.

2. The polynucleotide of claim 1, wherein the nucleotide deletion, addition and/or substitution result in altered expression of the variant CYP2B6 gene compared to the corresponding wild type gene.

3. A vector comprising the polynucleotide of claim 1 or 2.

4. The vector of claim 3, wherein the polynucleotide is operatively linked to expression control sequences allowing expression in prokaryotic or eukaryotic cells.

5. A host cell genetically engineered with the polynucleotide of claim 1 or 2 or the vector of claim 3 or 4.

6. A method for producing a molecular variant CYP2B6 protein or fragment thereof comprising
(a) culturing the host cell of claim 5; and
(b) recovering said protein or fragment from the culture.

7. A method for producing cells capable of expressing a molecular variant CYP2B6 gene comprising genetically engineering cells with the polynucleotide of claim 1 or 2 or the vector of claim 3 or 4.

8. A CYP2B6 protein or fragment thereof encoded by the polynucleotide of claim 1 or 2 or obtainable by the method of claim 6 or from cells produced by the method of claim 7.

9. An antibody which binds specifically to the protein of claim 8.

10. The antibody of claim 9 which specifically recognizes an epitope containing one or more amino acid substitution(s) as defined in claim 1.

11. A nucleic acid molecule complementary to a polynucleotide of claim 1 or 2.

12. A nucleic acid molecule capable of specifically recognizing and cleaving the polynucleotide of claim 1 or 2.

13. A vector comprising the nucleic acid molecule of claim 11 or 12.

14. A transgenic non-human animal comprising at least one polynucleotide of claim 1 or 2 or the vector of claim 3 or 4.

15. The transgenic non-human animal of claim 14 further comprising at least one inactivated wild type allele of the CYP2B6 gene.

16. The transgenic non-human animal of claim 14 or 15, which is a mouse or a rat.

17. A method of identifying and obtaining a CYP2B6 inhibitor capable of modulating the activity of a molecular variant of the CYP2B6 gene or its gene product comprising the steps of
(a) contacting the protein of claim 8 or a cell expressing a molecular variant CYP2B6 gene comprising a polynucleotide of claim 1 or 2 in the presence of components capable of providing a detectable signal in response to drug metabolization, with a compound to be screened under conditions to permit CYP2B6-mediated drug metabolization, and
(b) detecting the presence or absence of a signal or increase of a signal generated from the drug metabolization, wherein the presence or increase of the signal is indicative for a putative inhibitor.

18. The method of claim 17 wherein said cell is a cell of claim 5, obtained by the method of claim 7 or is comprised in the transgenic non-human animal of any one of claims 14 to 16.

19. A method of identifying and obtaining an CYP2B6 inhibitor capable of modulating the activity of a molecular variant of the CYP2B6 gene or its gene product comprising the steps of
(a) contacting the variant CYP2B6 protein of claim 8 with a first molecule known to be bound by wild type CYP2B6 protein to form a first complex of said protein and said first molecule;
(b) contacting said first complex with a compound to be screened; and
(c) measuring whether said compound displaces said first molecule from said first complex.

20. The method of claim 19, wherein said measuring step comprises measuring the formation of a second complex of said protein and said compound.

21. The method of claim 19 or 20, wherein said measuring step comprises measuring the amount of said first molecule that is not bound to said protein.

22. The method of any one of claim 19 to 21 wherein said first molecule is Cyclophosphamide, Iphosphamide, Tarnoxifen, Clopidogrel or Orphenadrine.

23. The method of any one of claims 19 to 22 wherein said first molecule is labeled.

24. A method of diagnosing a disorder related to the presence of a molecular variant of the CYP2B6 gene or susceptibility to such a disorder comprising
(a) determining the presence of a polynucleotide of claim 1 or 2 in a sample from a subject; and/or
(b) determining the presence of a protein of claim 8.

25. The method of claim 24, wherein said disorder is cancer.

26. The method of claim 24 or 25 comprising PCR, ligase chain reaction, restriction digestion, direct sequencing, nucleic acid amplification techniques, hybridization techniques or immunoassays.

27. The method of any one of claims 24 to 26, further comprising administering to a subject a medicament to abolish or alleviate said disorder.

28. The method of any one of claims 24 to 27, further comprising introducing
(i) a functional and expressible wild type CYP2B6 gene; and/or
(ii) a nucleotide acid molecule of claim 11 or 12 or the vector of claim 13 into cells.

29. A method for the production of a pharmaceutical composition comprising the steps of the method of any one of claims 17 to 23; and
(c) synthesizing and/or formulating the compound identified and obtained in step (b) or a derivative thereof in a pharmaceutically acceptable form.

30. A method for the preparation of a pharmaceutical composition comprising formulating a drug or pro-drug in the form suitable for therapeutic application and preventing or ameliorating the disorder of the subject diagnosed in the method of claim 24 or 25.

31. The method of claim 29 or 30 wherein said compound drug or prodrug is a derivative of a medicament as defined in claim 27.

32. An inhibitor identified or obtainable by the method of any one of claims 17 to 23.

33. The inhibitor of claim 32 which binds specifically to the protein of claim 8.

34. Use of an oligo- or polynucleotide for the detection of a polynucleotide of claim 1 or 2 and/or for genotyping of individual CYP2B6 alleles.

35. The use of claim 34 wherein said polynucleotide is a polynucleotide of claim 1 or 2 or a nucleic acid molecule of claim 11 or 12.

36. The use of claim 34 wherein said oligonucleotide is about 15 to 50 nucleotides in length and comprises the nucleotide sequence of any one of SEQ ID NOS: 1 to 43 or a complementary sequence.

37. A primer or probe consisting of an oligonucleotide as defined in claim 36.

38. Use of an antibody or a substance capable of binding specifically to the gene product of a CYP2B6 gene for the detection of the protein of claim 8, the expression of a molecular variant CYP2B6 gene comprising a polynucleotide of claim 1 or 2 and/or for distinguishing CYP2B6 alleles comprising a polynucleotide of claim 1 or 2.

39. A composition comprising the polynucleotide of claim 1 or 2, the vector of claim 3 or 4, the host cell of claim 5 or obtained by the method of claim 7, the protein of claim 8, the antibody of claim 9 or 10, the nucleic acid molecule of claim 11 or 12, the vector of claim 13, the inhibitor of claim 32 or 33 or the primer or probe of claim 37.

40. The composition of claim 39 which is a diagnostic or a pharmaceutical composition.

41. Use of an effective dose of a drug or prodrug for the preparation of a pharmaceutical composition for the treatment or prevention of a disorder of a subject comprising a polynucleotide of claim 1 or 2 in its genome.

42. The use of 41 wherein said disorder is cancer.


SEQUENCE LISTING (64 sequences)

SEQ ID NO: 1 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
acattcacttgctcacct

SEQ ID NO: 2 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gtaaataccacttgacca

SEQ ID NO: 3 - DNA, 24 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
atcctactcagaatgatgcacaac

SEQ ID NO: 4 - DNA, 24 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
attacaggtgagagtcatcacatc

SEQ ID NO: 5 - DNA, 19 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ggtctgcccatctataaac

SEQ ID NO: 6 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ctgattcttcacatgtctgcg

SEQ ID NO: 7 - DNA, 24 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tccctgggatttaactgtactcac

SEQ ID NO: 8 - DNA, 24 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
cagaattggcttggttggaatcta

SEQ ID NO: 9 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gacagaaggatgagggaggaa

SEQ ID NO: 10 - DNA, 22 bp, Artificial Sequence
  Description of Artificial Sequence artificial sequence
ctccctctgtctttcattctgt

SEQ ID NO: 11 - DNA, 23 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gtgattattcattaattgggttc

SEQ ID NO: 12 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgcaatggttgattgatgctc

SEQ ID NO: 13 - DNA, 23 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgagaatcagtggaagccataga

SEQ ID NO: 14 - DNA, 25 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
taattttcgataatctcactcctgc

SEQ ID NO: 15 - DNA, 19 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ataacagggtgcagaggca

SEQ ID NO: 16 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
aagtaccaaggcaagaagca

SEQ ID NO: 17 - DNA, 19 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ggctaattaccaatctggt

SEQ ID NO: 18 - DNA, 22 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
atatactcccttccctgatgca

SEQ ID NO: 19 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
actcagagccttcttccaact

SEQ ID NO: 20 - DNA, 24 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
acctgcatctctcagtgtttcatt

SEQ ID NO: 21 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
taggtgacagcctgatgttc

SEQ ID NO: 22 - DNA, 22 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tcatccttttctcgtgtgttct

SEQ ID NO: 23 - DNA, 23 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tctctccctgtgacctgctagct

SEQ ID NO: 24 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tcttctcacctctccatctt

SEQ ID NO: 25 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tatacacagcaaggctacag

SEQ ID NO: 26 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
atttctgcagctcagaagga

SEQ ID NO: 27 - DNA, 22 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gattacaggcatgagccaccat

SEQ ID NO: 28 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
attaagagaatccaggatgcc

SEQ ID NO: 29 - DNA, 22 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ttttgtggagtgtgtggagggt

SEQ ID NO: 30 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
ttctcaagttggggatagta

SEQ ID NO: 31 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
agagcgaagtgtatgcacct

SEQ ID NO: 32 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
agagccattgtctacagagg

SEQ ID NO: 33 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
acaatacctcactaagagtg

SEQ ID NO: 34 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gtctcagtttctgtctccttc

SEQ ID NO: 35 - DNA, 19 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgaacatgcactaccacca

SEQ ID NO: 36 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
atccttgcatgtgtatgagc

SEQ ID NO: 37 - DNA, 20 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgaaccaggagtagcaagag

SEQ ID NO: 38 - DNA, 23 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gtgattattcattaattgggttc

SEQ ID NO: 39 - DNA, 21 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgcaatggttgattgatgctc

SEQ ID NO: 40 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
acattcacttgctcacct

SEQ ID NO: 41 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
gtaaataccacttgacca

SEQ ID NO: 42 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
tgccgacatgtgatgtct

SEQ ID NO: 43 - DNA, 18 bp, Artificial Sequence
  Description of Artificial Sequence A primer for use in PCR amplification of the human genomic DNA to generate a polynucleotide which is capable of hybridizing to the CYP2B6 gene, and is useful for genotyping of individual CYP2B6 alleles.
aggtgagcaagtgaatgt

SEQ ID NO: 44 - DNA, 42 bp, Homo sapiens
agtgtcagatgacacagcacagcaagaccgaggcccttggtt

SEQ ID NO: 45 - DNA, 41 bp, Homo sapiens
agagacatatgggagtccagtagacatcaatcaaactggac

SEQ ID NO: 46 - DNA, 41 bp, Homo sapiens
caccagttctgcatctcttgctcctccttctgtttctccga

SEQ ID NO: 47 - DNA, 41 bp, Homo sapiens
gtacccaccatcacgcccggttaatttttgtgtttttagta

SEQ ID NO: 48 - DNA, 41 bp, Homo sapiens
gtggggaatggatgaaattttataacagggtgcagaggcag

SEQ ID NO: 49 - DNA, 41 bp, Homo sapiens
tcttgctactcctggttcagcgccaccctaacacccatgac

SEQ ID NO: 50 - DNA, 41 bp, Homo sapiens
ttcagcgccaccctaacacccatgaccgcctcccaccaggg

SEQ ID NO: 51 - DNA, 41 bp, Homo sapiens
ttcacggtacacctgggaccgaggcccgtggtcatgctgtg

SEQ ID NO: 52 - DNA, 41 bp, Homo sapiens
ggtgcatcagggaagggagtatatgggaggaagaaggactc

SEQ ID NO: 53 - DNA, 41 bp, Homo sapiens
acttcagtctgtgtccttgacctgctgcttcttcctagggg

SEQ ID NO: 54 - DNA, 41 bp, Homo sapiens
gaccccaccttcctcttccagtccattaccgccaacatcat

SEQ ID NO: 55 - DNA, 41 bp, Homo sapiens
aggcaagtttacaaaaacctgcaggaaatcaatgcttacat

SEQ ID NO: 56 - DNA, 41 bp, Homo sapiens
ctgcaggaaatcaatgcttacattggccacagtgtggagaa

SEQ ID NO: 57 - DNA, 41 bp, Homo sapiens
cgtgaaaccctggaccccagcgcccccaaggacctcatcga

SEQ ID NO: 58 - DNA, 39 bp, Homo sapiens
cctggaccccagcgcccccaaggacctcatcgacaccta

SEQ ID NO: 59 - DNA, 41 bp, Homo sapiens
tacccccaacataccagatccgcttcctgccccgctgaagg

SEQ ID NO: 60 - protein, 41 aa, Homo sapiens
ELSVLLFLALLTGLLLLLVQRHPNTHDRLPPGPRPLPLLGN

SEQ ID NO: 61 - protein, 41 aa, Homo sapiens
CLIEELRKSKGALMDPTFLFQSITANIICSIVFGKRFHYQD

SEQ ID NO: 62 - protein, 41 aa, Homo sapiens
QEINAYIGHSVEKHRETLDPSAPKDLIDTYLLHMEKEKSNA

SEQ ID NO: 63 - protein, 41 aa, Homo sapiens
NAYIGHSVEKHRETLDPSAPKDLIDTYLLHMEKEKSNAHSE

SEQ ID NO: 64 - protein, 25 aa, Homo sapiens
DIDLTPQECGVGKIPPTYQIRFLPR